(19) United States
(12) Patent Application Publication
(10) Pub. No.: US 2003/0050223 A1
(43) Pub. Date: Mar. 13, 2003

(54) Crystal forms and mutants of RANK ligand

(76) Inventors: Jonathan Lam, West Memphis, AR (US); F. Patrick Ross, Overland, MO (US); Steven L. Teitelbaum, St. Louis, MO (US); Christopher A. Nelson, St. Louis, MO (US); Daved H. Fremont, St. Louis, MO (US)

Correspondence Address: SONNENSCHEIN NATH & ROSENTHAL, P.O. BOX 061080, WACKER DRIVE STATION, CHICAGO, IL, 60606-1080, US

(21) Appl. No.: 10/215,446

(22) Filed: Aug. 9, 2002

Related U.S. Application Data
(60) Provisional application No. 60/311,163, filed on Aug. 9, 2001.

(51) Int. Cl. A61K 31/00; G01N 33/53; G01N 33/48; G06F 19/00; G01N 33/50; C07K 14/705

(52) U.S. Cl. 514/1; 530/350; 435/7.1; 702/19

(57) ABSTRACT

The present invention provides crystals of RANKL ectodomain complexes, detailed three dimensional structural information illustrating the interaction between RANKL and RANK and methods of utilizing the X-ray crystallographic data from such crystals and other structural data disclosed to design, identify and screen for compounds that modulate activity. The present invention also relates to machine readable media embedded with the three-dimensional atomic structure coordinates of RANKL crystalline complexes and subsets thereof, together with other structural data disclosed herein.

### CROSS-REFERENCE TO RELATED APPLICATIONS

This application is related to U.S. Application Serial No. 60/311,163, filed Aug. 9, 2001 and U.S. Application Serial No. 10/105,057, filed Mar. 22, 2002, which is related to U.S. Application Serial No. 60/277,855 filed Mar. 22, 2001 which are hereby incorporated by reference in their entirety.

### STATEMENT REGARDING FEDERALLY SPONSORED RESEARCH OR DEVELOPMENT

This invention was made in part with Government support under National Institutes of Health Grants AR32788, AR46523, AR46852, AR07033 and DE05413. The Government has certain rights in the invention.

### REFERENCE TO MICROFICHE APPENDIX

Not applicable.

### FIELD OF THE INVENTION

The present invention relates in general to methods for the treatment of osteoporosis and other bone deficiencies or defects using Receptor Activator of NFκB (RANK) Ligand and analogs or mimics thereof. More specifically, the invention relates to crystalline forms of the RANK Ligand (RANKL) protein, high-resolution X-ray diffraction structures, atomic structure coordinates, mutagenesis data, and three-dimensional structures obtained therefrom. The RANKL crystals of the invention and the structural information obtained by the applicants are useful for screening for, identifying and/or designing compounds that mimic or inhibit the ability of RANKL to interact with RANK and activate RANK signaling and in particular, modulate the biology of the differentiation and function of bone cells such as osteoblasts and osteoclasts, immune cells such as T and B cells and dendritic cells, mammary epithelium; and lymph node organogenesis, thereby treating, preventing, inhibiting or reversing (curing) diseases or conditions associated with the above-mentioned tissues.

### BACKGROUND OF THE INVENTION

The Tumor Necrosis Factor (TNF) family of cytokines plays an essential role in multiple biological functions including inflammation, organogenesis, host defense, autoimmunity, and apoptosis. The action of these potent biological mediators is achieved through a receptor-ligand interaction, leading to intracellular signaling and a change in cellular phenotype. The ligands exert their function by forming trimers and binding to their corresponding receptors. Subsequent receptor oligomerization results in conformational change of the receptor's intracellular domain, which then allows for members of the TNF receptor-associated factor (TRAF) family of adaptor proteins to bind and initiate a signaling cascade. TNF-R₂, TNF-R₁, TrkA, NGFR, CD₄₀, CD₃₀, OX-40, DR5, DR3, DR4 and RANK include some of the members of TNF receptor super-family that interact with different TRAF molecules (including 1-6) (Lewit-Bentley, A., et al., J. Mol. Biol. 199:389-392(1988), Banner, D. W., et al., Cel. 73:431-445(1993), Karpusas, M., et al., Structure. 3:1031-1039(1995), Hymowitz, S. G., et al., Mol. Cell. 4:563-571(1999), Mongkilsapaya, J., et al., Nat. Struc. Biol. 6:-1048-1053(1999), Cha, S. S., et al., J. Biol. Chem. 275:31171-31177(2000)).

The TNF family of receptors is characterized by cysteine-rich domains, which are located in the extracellular regions and are responsible for ligand binding. The crystal structures of TNFO, TNF, CD₄₀L, and TRAIL have shown that ligands, in their monomeric forms contain two -pleated sheets which assume a “jellyroll”-sandwich fold (Jones, E. Y., et al., Nature. 338:225-228(1989)). The ligands interact at hydrophobic surfaces to self-associate as noncovalent trimers that exist both as membrane-bound and soluble, cleaved forms (Idriss, H. T., et al., Microscopy Res. Tech. 50:184-195(2000)). The recognition between cognate ligands and receptors is highly specific and is characterized by dissociation constants in the nanomolar to picomolar range (Kim, D., et al., J. Exp. Med. 192:1467-1478(2000)).

Therapies modulating TNF super family proteins (SFPs) have been successfully utilized to treat conditions such as rheumatoid arthritis, inflammatory bowel disease and septicemia. Novel therapeutics that could have potential benefits for diseases such as cancer, autoimmune disorders, graft rejection, and bone-related disorders are also emerging.

The importance of these therapeutics can be appreciated in the light of the fact that bone-related disorders alone include a plethora of conditions such as osteoporosis, atherosclerosis, bone fracture or deficiency, periodontal disease, metastatic bone disease, and osteolytic bone disease. It is estimated that 30 million Americans and 100 million people worldwide are at risk for osteoporosis, the most common of these disorders (Mundy, et al., Science. 286:1946-1949 (1999)).

Bone remodeling and homeostasis are achieved through two distinct processes, known as bone synthesis and bone resorption. Bone synthesis is controlled by osteoblasts, bone stromal cells of mesenchymal origin, whose main function is to synthesize and deposit bone matrix and increase bone mass (Teitelbaum, S. L., Science. 289:1504-1508(2000), Kong, Y. Y., et al., Nature. 397:315-323(1999), Li, J., et al., Proc. Natl. Acad. Sci. U.S.A. 97:1566-1571(2000), Dougall, W. C., et al., Genes Dev. 13:2412-2424(1999), Kim, D., et al., J. Exp. Med. 192:1467-1478(2000)). The opposing process in which bone mass is lost (bone resorption) requires the action of osteoclasts. These cells are specialized, multinucleated macrophages, whose function includes resorption of both mature and newly synthesized bone.

RANKL, a transmembrane ligand expressed on osteoblasts, stromal cells, activated T cells, activated endothelium, and mammary epithelium has been found to interact with RANK, a transmembrane receptor on osteoclast precursor cells, initiating a signaling cascade that eventually leads to differentiation and maturation of osteoclast precursors to active osteoclasts. On the other hand, osteoclast development from its hematopoietic precursors can be prevented by binding of a decoy receptor osteoprotegerin (OPG) to RANKL, thereby preventing its interaction with RANK. The role of OPG was further confirmed by generating a knockout animal of this particular receptor. The phenotype exhibited by the OPG −/− mouse entailed severe osteoporosis, characterized by a dramatic decrease in strength and mineral density of bones.

Applicants have also discovered that, importantly, RANKL may be manipulated to stimulate osteogenesis, including enhanced activity of osteoblasts, commitment of osteoblast precursors to the osteoblast phenotype and bone matrix deposition (U.S. Patent Application Ser. No. 60/277,855). Such discovery provides the basis for methods useful to facilitate bone replacement or repair, as well as for treating diseases or conditions involving loss of bone mass by stimulating anabolic processes of bone formation.

RANKL is an essential cytokine in osteobiology. It mediates the differentiation of bone-absorbing osteoclasts from monocyte-macrophage precursors and modulates the survival and function of mature osteoclasts (Li, J., et al., Proc. Natl. Acad. Sci. U.S.A. 97:1566-1571(2000), Teitelbaum, S. L., Science. 289:1504-1508(2000)). Mice deficient in RANKL, or its receptor RANK, exhibit a complete lack of osteoclasts, resulting in severe osteopetrosis and hypocalcemia. Clinically, RANKL has been implicated in the pathogenesis of postmenopausal osteoporosis. In this disorder, estrogen deficiency leads to enhanced expression of TNF and RANKL, as well as decreased production of osteoprotegerin (OPG, a soluble TNFR family decoy receptor for RANKL) (Cenci, S., J. Clin. Invest. 106:1229-1237(2000), Hofbauer, L. C., et al., Endocrinol. 140:4367-4370(1999)). Together, these alterations in cytokine expression induce bone loss by stimulating osteoclast formation. We have recently demonstrated that TNF synergizes with RANKL both in vitro and in vivo to drive cells of the monocyte-macrophage lineage to differentiate along the osteoclastogenic pathway (Lam, J., et al., J. Clin. Invest. 106:1481-1488(2000)). This synergism is achieved at the level of AP-I and NFκB activation, two downstream transcriptional pathways activated by both TNFR and RANK, the signaling receptor for RANKL. RANKL also plays an essential role in immunobiology. Expressed on T cells, dendritic cells and their precursors, RANKL mediates the differentiation of T and B lymphocytes, as well as the survival of dendritic cells in the immune system. Hematopoietic CD4+ precursors require autocrine RANKL-RANK signaling for survival and differentiation while migrating from the marrow to primitive lymphoid tissue anlagen. Mice lacking RANKL or its receptor RANK exhibit defective peripheral and mesenteric lymph node organogenesis (Kong, Y. Y., et al., Nature. 397:315-323(1999), Dougall, W. C., et al., Genes Dev. 13:2412-2424(1999), Kim, D., et al., J. Exp. Med. 192:1467-1478(2000)). Clinically, the expression of RANKL by CD4+T cells mediates bone loss and joint destruction in animal models of arthritis and inflammatory osteolysis (Kong, Y. Y., et al., Nature. 402:304-309(1999), Teng, Y.-Y. A., et al., J. Clin. Invest. 106:R59-R67(2000)).

In addition to its regulatory roles in bone and the immune system, RANKL-RANK signaling also governs the differentiation of mammary epithelium (Fata, J. E., et al., Cell. 103:41-50(2000)). Pregnant mice lacking RANKL fail to form lactating breast tissue, with a complete absence of competent lobulo-alveolar glands. Mammary epithelial precursors lacking RANK undergo accelerated apoptosis due to a defect in Akt activation. Furthermore, the osteoclast deficit in these animals obviates mobilization of calcium from bone to the milk. Collectively, these observations illustrate the prominent role of RANKL-RANK in coordinating the normal development and function of lymphoid and mammary tissue, as well as the homeostatic regulation of calcium metabolism and bone mass.

Little is known about the atomic structure of most of the TNF super-family members. Similarly, little is known about the structural features of RANKL. Accordingly, a need exists to determine the crystal structure of RANKL, and related data useful for establishing its three-dimensional configuration in order to gain insight into the binding of this molecule to RANK and OPG. Crystal structure and related data would provide crucial information for synthesis and screening of potential mimics and analogs of RANKL which could then be applied to therapeutic purposes, in which modulation of RANK signaling is desired.

A number of crystal structures for TNF family cytokines, either alone or in complex with their receptors, have been reported—TNFα (Jones, E. Y., et al., Nature. 338:225-228(1989), Lewit-Bentley, A., et al., J. Mol. Biol. 199:389-392(1988)), TNFβ (Eck, M. J., et al., J. Bili. Chem. 267:2119-2122(1992)), TNFβ-TNFR complex (Banner, D. W., et al., Cell. 73:431-445(1993)), CD40L (Karpusas, M., et al., Structure. 3:1031-1039(1995)), ACRP30 (adipocyte complent-related protein) (Shapiro, L., et al., Curr. Biol. 8:335-338(1998)), TRAIL (Cha, S. S., et al., Immunity. 11:253-261(1999)), and the TRAIL-DR5 complex (Hymowitz, S. G., et al., 5. Mol. Cell. 4:563-571(1999), Mongkolsapaya, J., et al., Nat. Struct. Biol. 6:1048-1053(1999), Cha, S. S., et al., J. Biol. Chem. 275:31171-31177(2000)). From these analyses, it is evident that the amino acid conservation among TNF cytokines is largely confined to internal hydrophobic residues involved in trimer assembly. In contrast, the unique regions form surface loops of varying composition and length that connect core β-strands. Importantly, however, the overall lack of sequence conservation among both ligands and receptors precludes meaningful structural prediction.

Accordingly, a need exists, in general, for designing new compositions and methods for preventing or inhibiting bone loss or for enhancing bone formation, particularly wherein bone resorption is not otherwise affected. However, identification of such compositions has heretofore relied on serendipity and/or screening of large numbers of natural and synthetic compounds. A far superior method of drug screening relies on structure-based drug design. The three-dimensional structures of proteins or protein fragments relevant to bone formation if determined could lead to the design of potential agonists and/or potential antagonists with the aid of computer modeling. However, heretofore the role of RANKL in bone formation and the three-dimensional structure of this important protein were unknown.

Therefore, there is presently a need for obtaining three-dimensional structural information, including data generated from crystals of RANKL protein to allow such design data to be obtained. Finally, there is a need for procedures for related structural based drug design based on such crystallographic and atomic structural data.

### BRIEF SUMMARY OF THE INVENTION

Accordingly, the present invention provides many crystalline forms of polypeptides that constitute the biologically active ectodomain of RANKL. Thus, further objects of the present invention include the provision of the atomic structure coordinates obtained from the crystals, the determination of the location of RANKL's unique solvent-accessible surface loops, the specific location of RANKL's intersubunit hydrogen bonds and the location of RANKL's homotrimer intersubunit grooves required for receptor trimerization and methods of utilizing the three-dimensional structural information obtained by applicants to design or identify compounds which enhance or inhibit bone formation activity. Another related object is to provide machine- or computer-readable media embedded with the three-dimensional structural information obtained by applicants, or portions or subsets thereof which can be used to identify or design bone forming or bone formation and resorption inhibiting compounds. A further object is to provide methods of making the crystals of the invention.

In another aspect, the invention provides compositions comprising crystalline forms of polypeptides corresponding to RANKL ectodomain complexes of crystallized polypeptides.

Among others, certain of the RANKL homotrimer crystals are characterized by a spacegroup of either P2₁2₁2₁, and a unit cell of a=65.3 Å±0.2 Å, b=82.0 Å±0.2 Å, c=99.5 Å±0.2 Å, or spacegroup R3 and a unit cell of a=b=150.6 Å±0.2 Å and c=139.5 Å±0.2 Å and are preferably of diffraction quality. In a preferred embodiment, the crystals are of sufficient quality to permit the determination of the three-dimensional X-ray diffraction structure of the crystalline polypeptide to high resolution, preferably to a resolution of at least about 3 Å to 2.6 Å, typically in the range of about 1 Å to about 3 Å.

The invention also provides methods of making the crystals of the invention. Generally, crystals of the invention are grown by dissolving substantially pure polypeptides in an aqueous buffer that includes a precipitant at a concentration just below that necessary to precipitate the polypeptide. Water is then removed by controlled evaporation to produce precipitating conditions, which are maintained until crystal growth ceases.

In another aspect, the invention provides machine- or computer-readable media embedded with the three-dimensional structural information obtained from the RANKL crystals of the invention, or portions or subsets thereof. Such three-dimensional structural information will typically include the atomic structure coordinates of the crystallized polypeptide, or the atomic structure coordinates of a portion thereof, such as, for example, the atomic structure coordinates of an active or binding site, but may include other structural information, such as vector representations of the atomic structure coordinates, etc.

Thus, the atomic structure coordinates and machine-readable media of the invention have a variety of uses. As such, provided are methods of identifying, among other potential uses, bone-forming compounds which utilize the coordinates for solving the three-dimensional X-ray diffraction and/or solution structures of other proteins, including mutant forms, to high resolution. Structural information may also be used in a variety of molecular modeling and computer-based screening applications to, for example, intelligently design mutants of the crystallized RANKL having altered biological activity and to computationally design and identify compounds that bind RANKL, its receptors, or a portion or fragment of RANKL or its receptors.

In another aspect, the present invention provides methods of using the coordinates of the RANKL crystal, or subsets of such structure coordinates, to design or identify candidate compounds capable of modulating RANK biological activity. Such candidate compounds may be evaluated for biological activity, such as, for example, the ability to bind (preferably competitively) a subunit of interest or the ability to disrupt receptor signaling, or the ability to disrupt binding. In one embodiment, the crystals from which the RANKL structure is derived have the space group and cell dimensions described above, such that the three dimensional structure of the co-complex is provided to a resolution of from about 3.0 Å to about 2.6 Å or greater precision.

In a further aspect of the invention, such potential compounds are evaluated for biological activity. Candidate compounds are designed or identified using the atomic structure coordinates of the RANKL complexes or subsets thereof, synthesized and screened for their ability to enhance osteoblast proliferation or other osteogenetic activity, thereby increasing bone formation (or inhibiting or preventing bone loss). The bone forming activity of the compound is determined by in vitro and animal model assays as described in detail in U.S. Application Serial No. 60/277,855.

Other objects and features will be in part apparent and in part pointed out hereinafter.

### BRIEF DESCRIPTION OF THE SEVERAL VIEWS OF THE DRAWINGS

FIG. 1: Crystallographic data and refinement statistics for murine RANKL.

FIG. 2A: Stereo ribbon diagram of the RANKL trimer. Shown with the β-strands and connecting loops of one RANKL monomer labeled according to standard TNF β-sandwich nomenclature. The other two RANKL monomers appear in two different shades.

FIG. 2B: Stereo ribbon diagram of the RANKL trimer. In this view, oriented identically to FIG. 2A, the RANKL transmembrane stalk projects to the top of the image, while the membrane-distal region is toward the bottom. The homotrimer exhibits the shape of a truncated pyramid, being slightly wider at the membrane proximal end.

FIG. 2C: Stereo ribbon diagram of the RANKL trimer. Ribbon diagram of the RANKL trimer viewed down the axis of three-fold symmetry, oriented with the membrane-distal face forward. The secondary structure of monomer X is labeled as in FIG. 2A.

FIG. 2D: Stereo ribbon diagram of the RANKL trimer. The RANKL trimer, shown with the molecular surfaces of monomers X, Y, and Z appearing as three different shades.

FIG. 2E: Stereo ribbon diagram of the RANKL trimer. Comparison of a single RANKL monomer with those of TNF and TRAIL. The β-strands of all three structures appear in the same shade, while the connecting loops appear in different shades: lightest for RANKL, medium for TNF (pdb code 1tnr) and darkest for TRAIL (pdb code 1d4v). When the structures of these proteins are superimposed, it is apparent that the β-strands of RANKL superimpose almost identically with those of TNF and TRAIL. In contrast, the AA″, CD, EF, and DE loops of RANKL exhibit unique topology when compared with those of other TNF family cytokines.

FIG. 2F: Stereo ribbon diagram of the RANKL trimer. Electron density of the E-D-G β-strands of the RANKL monomer. The structure is viewed from the solvent-accessible surface of the RANKL monomer, with an orientation similar to FIG. 2E. Displayed in different shades is a 2.6 Å composite omit map (contoured at 1.2) with the RANKL structure showing carbon, oxygen, nitrogen and sulfur.

FIG. 3 is a structure-based alignment of TNF family cytokines with RANKL. The sequence of the extracellular core domains of the TNF family cytokines TRAIL, CD₄₀L, TNFα, TNFβ, and ACRP₃₀ are shown aligned to that of murine RANKL. Structural alignments to RANKL are based upon pairwise topological residue superposition of the crystal structure of RANKL with those of TRAIL (1d4v), CD₄₀L (1aly), TNFα (2tnf), TNFβ (1tnr), and ACRP₃₀ (1c28). Residue numbers and secondary structure assignments for RANKL are depicted above the sequences. The 10 β-strands that constitute the TNF family β-sandwich are drawn as medium shaded arrows, with standard nomenclature. Solvent accessible loops and coil regions connecting the β-strands are illustrated as darker shaded lines. The AA″ loop (light shaded line), connecting β-strands A and A″ encompasses the A″ β-strand. Dark shaded triangles above the residues indicate those involved in the trimeric interface of RANKL. The degree of homology at structurally equivalent positions among the family members is shown by colored circles below the sequences, 0-50% conservation (no circles), 50-90% (light shaded), >90% (dark shaded). TRAIL residues boxed denote DR5 receptor contact sites on TRAIL. TNF residues boxed identify TNF receptor contact sites on TNF. By substituting RANKL in place of the ligands in the TRAIL:DR5 and TNF:TNFR co-crystal structures, we calculated potential contact sites for both of these receptors on RANKL. Boxed residues of RANKL indicate those calculated to physically contact DR5 (medium shaded), TNFR (dark shaded), or both (light shaded) receptors during the docking analysis. Structural elements that are unique to RANKL are clustered in the solvent-accessible AA″, CD, DE, and EF loops.

FIGS. 4A: Receptor regions of RANKL. Schematic depiction of RANKL trimer (lighter, medium and darker shaded monomers) docked with its receptor RANK (drawn here as four cysteine-rich domains, very light shaded).

FIGS. 4B: Receptor regions of RANKL. Surface representation of a RANKL trimer, oriented as in FIG. 4A, docked with a monomer of DR5, the Trail receptor (light shaded C backbone worm). Areas on the surface of RANKL calculated to engage DR5 appear in medium shade, as on the sequence in FIG. 3.

FIGS. 4C: Receptor regions of RANKL. Surface representation of a RANKL trimer docked with a monomer of the TNF receptor (light shaded C backbone worm). Areas on the surface on RANKL calculated to engage the TNFR appear in medium shade.

FIGS. 4D: Receptor regions of RANKL. Comparison of DR5 (medium shading) and TNFR (darker shading) contact sites on the surface of RANKL. Overlapping areas that contact both receptors appeal in light shade.

FIGS. 4E: Receptor regions of RANKL. Surface representation of a RANKL trimer with degrees of conservation mapped to the surface. The degree of conservation is directly proportional to intensity of colorization (light=no conservation, dark=conserved). Areas that contact both receptors show little to no sequence conservation across this family of cytokines (light shades). These unique regions provide specificity for receptor-ligand recognition, underlying the lack of cross-reactivity among receptors and ligands in this cytokine family.

FIGS. 4F: Receptor regions of RANKL. Potential receptor contact areas of RANKL that were targeted for structure-based mutagenesis appear in dark shade.

FIG. 5A: Structure-based mutagenesis of RANKL. Ribbon view of AA″ loop illustrating the AA″ loop deletion and AA″ loop swap mutants. For the AA″ loop deletion, SGSHKVTLSS was mutated to SSS by removal of the segment appearing in dark shade (GSHKVTL). For the AA″ loop swap mutant, SGSHKVTLSS was mutated to SLL by swapping the segments depicted in light and dark shades with the analogous segments from TNF.

FIG. 5B: Structure-based mutagenesis of RANKL. Dose response curves for native and mutant forms of RANKL (filled square—native RANKL; filled circle—I₂₄₈D point substitution; filled triangle—AA″ loop deletion; open triangle—AA″ loop swap) are plotted for in vitro osteoclastogenesis. ED₅₀ of native RANKL is ˜12 ng/ml, compared with ˜100 ng/ml for I₂₄₈D RANKL. AA″ loop deletion and AA″ loop swap mutants failed to induce detectable osteoclastogenesis at any dosage.

FIG. 5C: Structure-based mutagenesis of RANKL. Osteoclastogenic cultures are depicted at identical magnification following 7 days of exposure to the various forms of RANKL. Cultures were stained for osteoclast-specific tartrate-resistant acid phosphatase (TRAP) activity (dark shaded reaction product). Native RANKL induces the formation of large, multinucleated, TRAP-expressing osteoclasts. I₂₄₈D RANKL generated TRAP-positive cells that lack the characteristic morphology of osteoclasts, with the majority of cells exhibiting mononuclearity. AA″ loop deletion and loop swap mutants fail to induce differentiation along the osteoclastogenic pathway.

More detailed descriptions of the Figures are contained in the Figure Legends below, as well as in relevant portions of the following specification.

### ABBREVIATIONS AND DEFINITIONS

To facilitate understanding of the invention, a number of terms are defined below:

The amino acid notations used herein for the twenty genetically encoded L-amino acids are conventional and are abbreviated as follows:

TABLE 1

| | One-Letter | Three-Letter |
|---|---|---|
| Amino Acid | Symbol | Symbol |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

As used herein, unless specifically delineated otherwise, the three-letter and one-letter amino acid abbreviations designate amino acids in either the D-configuration or the L-configuration. For example, Arg designates D-arginine and L-arginine, and R designates D-arginine and L-arginine.

Unless noted otherwise, when polypeptide sequences are presented as a series of one-letter and/or three-letter abbreviations, the sequences are presented in the N→C direction, in accordance with common practice. As used herein, “C” refers to the alpha carbon of an amino acid residue.

For purposes of determining conservative amino acid substitutions in the various polypeptides described herein and for describing the various peptide and peptide analog compounds, the amino acids can be conveniently classified into two main categories—hydrophilic and hydrophobic—depending primarily on the physical-chemical characteristics of the amino acid side chain. These two main categories can be further classified into subcategories that more distinctly define the characteristics of the amino acid side chains. For example, the class of hydrophilic amino acids can be further subdivided into acidic, basic and polar amino acids. The class of hydrophobic amino acids can be further subdivided into non-polar and aromatic amino acids. The definitions of the various categories of amino acids are as follows:

“Hydrophilic amino acid” refers to an amino acid exhibiting a hydrophobicity of less than zero according to the normalized consensus hydrophobicity scale of Eisenberg, et al., J. Mol. Biol. 179:125-142(1984). Genetically encoded hydrophilic amino acids include Thr (T), Ser (S), His (H), Glu (E), Asn (N), Gin (Q), Asp (D), Lys (K) and Arg (R).

“Acidic amino acid” refers to a hydrophilic amino acid having a side chain pK value of less than 7. Acidic amino acids typically have negatively charged side chains at physiological pH due to loss of a hydrogen ion. Genetically encoded acidic amino acids include Glu (E) and Asp (D).

“Basic amino acid” refers to a hydrophilic amino acid having a side chain pK value of greater than 7. Basic amino acids typically have positively charged side chains at physiological pH due to association with hydronium ion. Genetically encoded basic amino acids include His (H), Arg (R) and Lys (K).

“Polar amino acid” refers to a hydrophilic amino acid having a side chain that is uncharged at physiological pH, but which has at least one bond in which the pair of electrons shared in common by two atoms is held more closely by one of the atoms. Genetically encoded polar amino acids include Asn (N), Gin (Q) Ser (S) and Thr (T).

“Hydrophobic amino acid” refers to an amino acid exhibiting a hydrophobicity of greater than zero according to the normalized consensus hydrophobicity scale of Eisenberg, et al., J. Mol. Biol. 179:125-142(1984). Genetically encoded hydrophobic amino acids include Pro (P), Ile (I), Phe (F), Val (V), Leu (L), Trp (W), Met (M), Ala (A), Gly (G) and Tyr (Y).

“Aromatic amino acid” refers to a hydrophobic amino acid with a side chain having at least one aromatic or heteroaromatic ring. The aromatic or heteroaromatic ring may contain one or more substituents such as —OH, —SH, —CN, —F, —Cl, —Br, —I, —NO₂, —NO, —NH₂, —NHR, —NRR, —C(O)R, —C(O)OH, —C(O)OR, —C(O)NH₂, —C(O)NHR, —C(O)NRR and the like where each R is independently (C₁-C₆) alkyl, substituted (C₁-C₆) alkyl, (C₂-C₆) alkenyl, substituted (C₂-C₆) alkenyl, (C₂-C₆) alkynyl, substituted (C₂-C₆) alkynyl, (C₅-C₂₀) aryl, substituted aryl, (C₆-C₂₆) arylalkyl, substituted (C₆-C₂₆) arylalkyl, 5-20 membered heteroaryl, substituted 5-20 membered heteroaryl, 6-26 membered heteroarylalkyl or substituted 6-26 membered heteroarylalkyl. Genetically encoded aromatic amino acids include His (H), Phe (F), Tyr (Y) and Trp (W).

“Apolar amino acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded apolar amino acids include Leu (L), Val (V), Ile (I), Met (M), Gly (G) and Ala (A).

“Aliphatic amino acid” refers to a hydrophobic amino acid having an aliphatic hydrocarbon side chain. Genetically encoded aliphatic amino acids include Ala (A), Val (V), Leu (L) and Ile (I).

“Hydroxyl-substituted aliphatic amino acid” refers to a hydrophilic polar amino acid having a hydroxyl-substituted side chain. Genetically encoded hydroxyl-substituted aliphatic amino acids include Ser (S) and Thr (T).

The amino acid residue Cys (C) is unusual in that it can form disulfide bridges with other Cys (C) residues or other sulfanyl-containing amino acids. The ability of Cys (C) residues (and other amino acids with —SH containing side chains) to exist in a peptide in either the reduced free —SH or oxidized disulfide-bridged form affects whether Cys (C) residues contribute net hydrophobic or hydrophilic character to a peptide. While Cys (C) exhibits a hydrophobicity of 0.29 according to the normalized consensus scale of Eisenberg (Eisenberg, supra), it is to be understood that for purposes of the present invention Cys (C) is categorized as a polar hydrophilic amino acid, notwithstanding the general classifications defined above.

As will be appreciated by those of skill in the art, the above-defined categories are not mutually exclusive. Thus, amino acids having side chains exhibiting two or more physical-chemical properties can be included in multiple categories. For example, amino acid side chains having aromatic moieties that are further substituted with polar substituents, such as Tyr (Y), may exhibit both aromatic hydrophobic properties and polar or hydrophilic properties, and can therefore be included in both the aromatic and polar categories. As another example, His (H) has a side chain that falls within the aromatic and basic categories. The appropriate categorization of any amino acid will be apparent to those of skill in the art, especially in light of the detailed disclosure provided herein.

While the above-defined categories have been exemplified in terms of the genetically encoded amino acids, the amino acid substitutions need not be, and in certain embodiments preferably are not, restricted to the genetically encoded amino acids. Indeed, since many of the compounds described herein may be produced synthetically, they may comprise one or more genetically non-encoded amino acids. Thus, in addition to the naturally occurring genetically encoded amino acids, amino acid residues in the core peptides of structure (I) may be substituted with naturally occurring non-encoded amino acids and synthetic amino acids.

Certain commonly encountered amino acids of which the compounds of the invention may be comprised include, but are not limited to,. -alanine (-Ala) and other omega-amino acids such as 3-aminopropionic acid, 2,3-diaminopropionic acid (Dpr), 4-aminobutyric acid and so forth; -aminoisobutyric acid (Aib);.-aminohexanoic acid (Aha);.-aminovaleric acid (Ava); N-methylglycine or sarcosine (MeGly); ornithine (Orn); citrulline (Cit); t-butylalanine (t-BuA); t-butylglycine (t-BuG); N-methylisoleucine (Melle); phenylglycine (Phg); cyclohexylalanine (Cha); norleucine (Nle); naphthylalanine (Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic).;-2-thienylalanine (Thi); methionine sulfoxide (MSO); homoarginine (hArg); N-acetyl lysine (AcLys); 2,4-diaminobutyric acid (Dbu); 2,3-diaminobutyric acid (Dab); p-aminophenylalanine (Phe(pNH₂)); N-methyl valine (MeVal); homocysteine (hCys), homophenylalanine (hPhe) and homoserine (hSer); hydroxyproline (Hyp), homoproline (hPro), N-methylated amino acids and peptoids (N-substituted glycines).

The classifications of the genetically encoded and common non-encoded amino acids according to the categories defined above are summarized in Table 2, below. It is to be understood that Table 2 is for illustrative purposes only and does not purport to be an exhaustive list of amino acid residues that can be used in the invention. Additional amino acids may be found in Fasman, 1989, Practical Handbook of Biochemistry and Molecular Biology, CRC Press, Inc., pp. 3-70, and the references cited therein.

TABLE 2
CLASSIFICATIONS OF COMMONLY ENCOUNTERED AMINO
ACIDS

| | Genetically | Non-Genetically |
|---|---|---|
| Classification | Encoded | Encoded |
| Hydrophobic | | |
| Aromatic | H, F, Y, W | Phg, Nal, Thi, Tic, Phe(4- |
| | | Cl), Phe-(2-F), Phe(3-F), Phe(4- |
| | | F), hPhe |
| Apolar | L, V, I, M, G, A, P | t-BuA, t-BuG, Melle, Nle, |
| | | MeVal, Cha, MeGly, Aib |
| Aliphatic | A, V, L, I | b-Ala, Dpr, Aib, Aha, MeGly, t- |
| | | BuA, t-BuG, Melle, Cha, Nle |
| | | MeVal |
| Hydrophilic | | |
| Acidic | D, E | |
| Basic | H, K, R | Dpr, Orn, hArg, Phe(p-NH₂), |
| | | Dbu, Dab |
| Polar | C, Q, N, S, T | Cit, AcLys, MSO, bAla, hSer |

The phrase “preventing or inhibiting” is being affected either by being inhibited, expressed in another manner, or reduced to such an extent that the observed property is measurably lower than is the case when the treatment is not employed. Measurement of the degree of inhibition can be determined in vitro by methods known to the person skilled in the art.

The compounds and compositions of the present invention which mimic RANKL are said to exhibit “RANKL activity.”

By the term “subject in need thereof” is in the present context meant a subject, which can be any animal, including a human being, who is in need of treatment. By the term “an effective amount” is meant an amount of the substance in question which will in a majority of patients have either the effect that the disease is cured or ameliorated or, if the substance has been given prophylactically, the effect that the disease is prevented from manifesting itself. The term “an effective amount” also implies that the substance is given in an amount which only causes mild or no adverse effects in the subject to whom it has been administered, or that the adverse effects may be tolerated from a medical and pharmaceutical point of view in the light of the severity of the disease for which the substance has been given.

As used herein, “treatment” includes both prophylaxis and therapy. Thus, in treating a subject, the compounds of the invention may be administered to a subject already harboring a condition or disease or in order to prevent such condition or disease from occurring.

By the term “a mimic of RANKL” is meant a compound which has been established to have the ability of binding to and activating RANK.

In the present context the terms “an analog of RANKL” and “a mimic of RANKL” should be understood, in a broad sense, to mean any substance which mimics (with respect to structural characteristics) an effective part of the ectodomain of RANKL. Thus, the analog or mimic may simply be any other compound regarded as capable of mimicking the binding between RANKL and RANK or OPG in vivo or in vitro.

The term “crystallized ectodomain of RANKL” refers to a polypeptide complex having a complete or functional fragment of the amino acid sequence of the extracellular domain of RANKL or a homologous sequence and which is in crystalline form.

The term “crystal” refers to a composition comprising a polypeptide in crystalline form. The term “crystal” includes native crystals, heavy-atom derivative crystals and co-crystals, as defined herein.

The term “native crystal” refers to a crystal wherein the polypeptide is substantially pure. As used herein, native crystals do not include crystals of polypeptides comprising amino acids that are modified with heavy atoms, such as crystals of selenomethionine mutants, selenocysteine mutants, etc.

The term “heavy-atom derivative crystal” refers to a crystal wherein the polypeptide is in association with one or more heavy-metal atoms. As used herein, heavy-atom derivative crystals include native crystals into which a heavy metal atom is soaked as well as crystals of selenomethionine mutants and selenocysteine mutants.

The term “co-complex” refers to a polypeptide in association with one or more additional polypeptides or other molecules. For example, the ectodomain of RANKL in association with another polypeptide such as RANK or OPG.

The term “co-crystal” refers to a composition comprising a co-complex, as defined above, in crystalline form. Co-crystals include native co-crystals and heavy-atom derivative co-crystals.

The term “unit cell” refers to the smallest and simplest volume element (i.e., parallelpiped-shaped block) of a crystal that is completely representative of the unit or pattern of the crystal. The dimensions of the unit cell are defined by six numbers: dimensions a, b and c and angles α, β, and γ (Blundel, et al., Protein Crystallography. Academic Press (1976)). A crystal is an efficiently packed array of many unit cells.

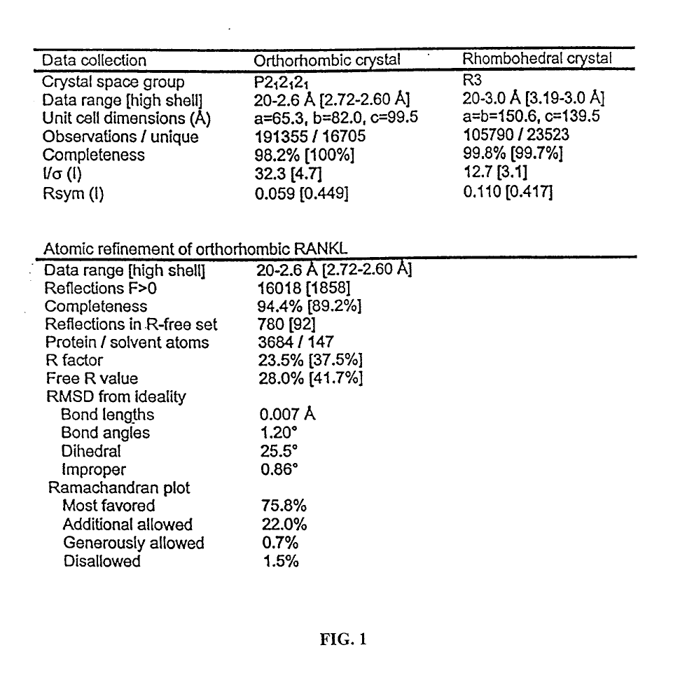

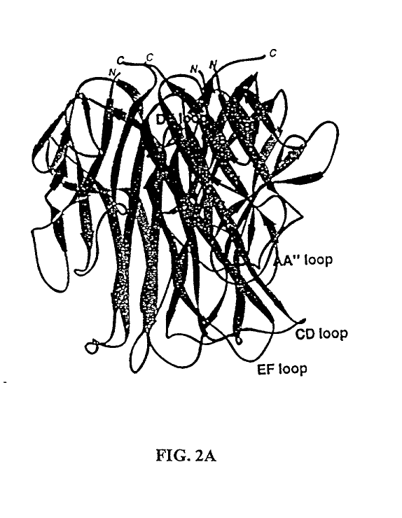

The phrase “having substantially the same three-dimensional structure” refers to a polypeptide that is characterized by a set of atomic structure coordinates that have a root mean square deviation (r.m.s.d.) of less than or equal to about 3 Å when superimposed onto the atomic structure coordinates of FIG. 1 when at least about 50% to 100% of the C atoms of the coordinates are included in the superposition.

### DETAILED DESCRIPTION OF THE INVENTION

In accordance with the present invention, applicants have provided three-dimensional -structural data essential for designing and fabricating compounds which mimic the biologically essential regions of RANKL which thereby function to activate RANK. Specifically, applicants have created crystalline forms of the ectodomain of RANKL protein, and prepared high-resolution X-ray diffraction analyses, atomic structure coordinates, mutagenesis data, and three-dimensional structures obtained therefrom which may be used to devise compounds and methods which mimic or inhibit the activity of RANKL.

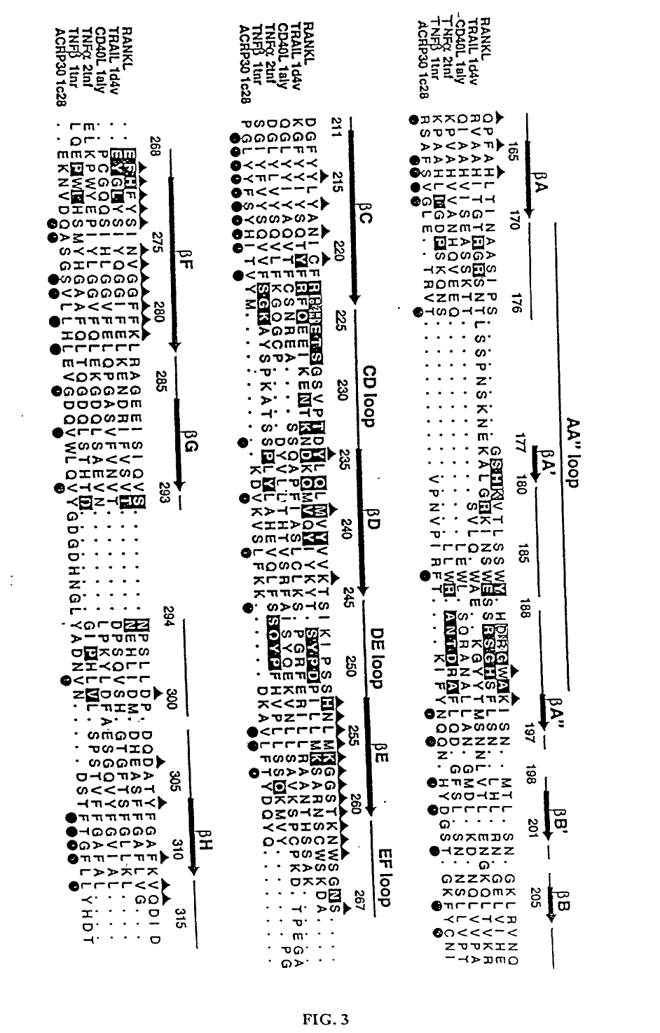

The crystal structure of the ectodomain of RANKL has been resolved and refined to a 2.6 Å resolution, revealing molecules self-assembled into stable, noncovalently associated trimers. The residues involved in this trimerization are indicated in FIG. 3. A notable feature of the RANKL trimer interface is the presence of a conserved ring of hydrophobic side chains lining the core at the base of the molecule. This aromatic tiling, commonly observed in TNF family members, constitutes a major driving force for self-assembly. In RANKL, a number of conserved residues contribute to this tiling -including His₁₆₇, Tyr₂₁₄, Tyr₂₁₆, Phe₂₈₀, and Phe₃₁₀. Surprisingly, 70% of the trimer contacts are nonconserved at the amino acid level. Some of these residues structurally preclude hetero-association with other TNF family cytokines, such as: Trp₁₉₂ and Lys₁₉₄ in the AA″ loop; Trp₂₆₃ in the EF loop; and three residues of the F beta-strand, Phe₂₆₉, His₂₇₀, and Phe₂₇₁. The majority of the unique trimer contacts of RANKL are polar in nature. RANKL is unusual among the TNF family in employing 46 buried polar interactions at the trimer interface, twice the number observed in a typical TNF family cytokine. These polar interactions are sequence-specific, thereby contributing to the specificity of self-association. Notable charged interactions between neighboring monomers include Lys,₁₉₄-Asp₃₀₁, Lys₂₄₃-Asp₂₉₉, and Lys₂₅₆-Asp₃₀₃.

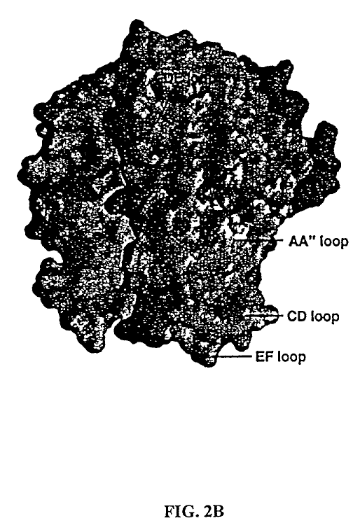

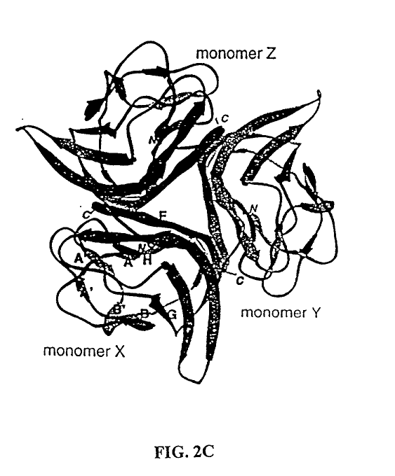

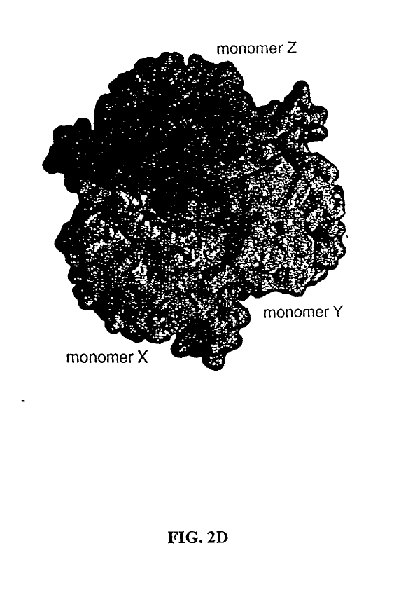

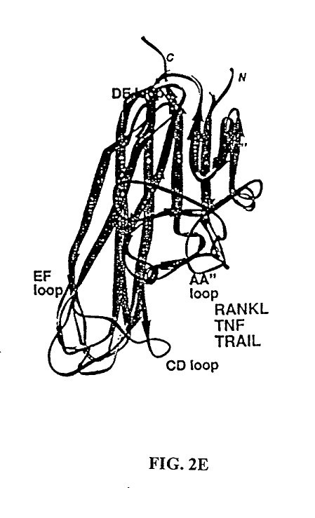

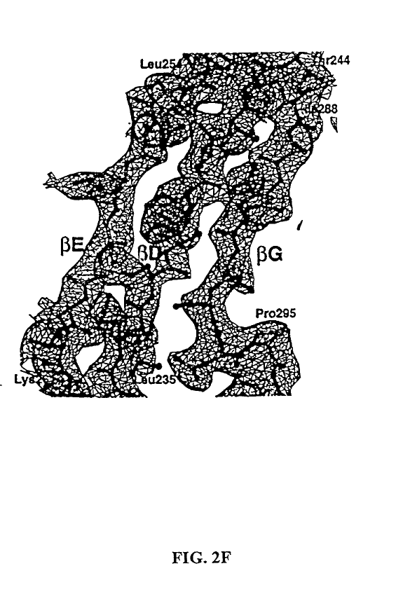

RANKL amino acid diversity is most extreme in the surface exposed loops and flanking strands, in which little sequence and structural homology to the family can be discerned. As shown in FIG. 2e, the AA″ loop of TRAIL traverses horizontally across the face of the monomer, and shorter AA″ loop of TNF follows a tight hairpin turn to immediately join the A″ beta-strand, the AA″ loop of RANKL projects towards the apex of the molecule. The AA″ loop, together with the displacement of the CD loop, confers a unique surface to the upper third of the RANKL molecule. A subtler outward shift of the DE loop shapes the receptor-binding groove at the base of the RANKL molecule. This suggests that sequence diversity localized at the receptor-binding interfaces of the ligand, and complementary diversity of residues in the receptor is a major determinant of specificity among TNF family members.

Applicants used the structures of the two known TNF family receptor-ligand complexes, TNFβ-TNFR and TRAIL-DR5, to provide a biological context for modeling the interaction of RANKL with RANK. Applicants identified prospective receptor contact residues using docking analyses with these structures. The biological relevance of these contact residues—Lys₁₈₀, Asp₁₈₉, and Arg₁₉₀ in the AA″ loop; His₂₂₃ and His₂₂₄ in the CD loop; and Lys₂₄₇, Ile₂₄₈, and Pro₂₄₉ in the DE loop—were confirmed by performing structure-based mutagenesis experiments. A single amino acid substitution in the DE loop significantly lowers the potency of RANKL and substantially suppresses its ability to induce the differentiation of morphologically sound osteoclasts. Also, the deletion of the AA″ loop and its substitution with the analogous loop of TNF leave the core topology of RANKL intact, since this protein readily and spontaneously self-assembles as homotrimers. However, the inability of these RANKL mutants to demonstrate biological activity establishes the necessity of the AA″ loop for RANK activation. As detailed herein, applicants' structural and functional analysis provides a method for the rational pharmacological modulation of RANKL and its receptors for therapeutic purposes.

As elucidated, RANKL is a type II transmembrane protein containing a membrane-anchoring domain, a connecting stalk, and a receptor-binding ectodomain. Similar to all TNF cytokines, RANKL has a structural core consisting of 10 hydrogen-bonded strands, in two sheets, that assume a characteristic jellyroll β-sandwich fold. RANKL differs from other TNF cytokines in the length and composition of its surface loops that connect the beta-strands. During synthesis, RANKL monomers self-assemble into noncovalently associated trimers. The core beta-strand topology underlies the intrinsic nature of RANKL, like other cytokines, to oligomerize around an axis of three-fold rotational symmetry. Biologically active trimers of RANKL can exist as both membrane-bound and soluble, cleaved forms.

Although the receptors for RANKL, RANK and OPG, have been categorized as members of the TNF super-family according to sequence homology, no three-dimensional structures have been reported to date. Expressed as type I transmembrane proteins, all TNF receptors are thought to employ a tertiary folding pattern to form elongated structures that project from the cell membrane. The extracellular ligand-binding domains are constructed primarily of three types of small, modular cysteine rich repeats. In general, the amino terminal cysteine-rich domain (CRD) functions as a pre-ligand assembly domain (PLAD), and the second and third CRDs function in ligand binding. Ligation of TNF family receptors by their cognate ligands induces the cytoplasmic tails to form signaling complexes with a characteristic 3-fold stoichiometry, allowing TNF receptor-associate factors (TRAFs) or Fas-associated with death domain (FADD) proteins to activate downstream effector pathways, including MAP (mitogen-activated protein) kinases, NFκB, and Phosphatidylinositol-3-Kinase (PI3K).

Recognition between TNF family cytokines and their receptors is highly specific, with typical dissociation constants in the nanomolar to picomolar range. This specificity is underscored by the fact that no hetero-trimerization among TNF family cytokines has been observed, even though individual cells often express more than one family member.

Applicants have determined that unique regions of RANKL form surface loops which differ from other TNF members as to their composition and length. These unique loops connect the core beta-strands. Importantly, applicants have elucidated these unique structural motifs of RANKL despite the overall lack of sequence conservation among TNF ligands and receptors which precluded meaningful structural prediction.

Applicants successfully formed RANKL crystals, resolving the crystal structure of complexes of the ectodomain of the murine RANKL to 2.6 Å, determined that RANKL adopts the characteristic structural fold of a TNF family cytokine, and identified elements of RANKL that interact with its receptors. Applicants then confirmed the necessity of these elements in mediating the ability of RANKL to activate RANK by forming RANKL mutants.

### Structure Determination

The biologically active ectodomain of murine RANKL (residues 158-316, QPFA . . . QDID), expressed recombinantly in E. coli, crystallizes in two forms: a rhombohedral crystal containing seven monomers in the ASU, arranged as two trimers with a seventh monomer situated on the crystallographic three-fold axis of symmetry; and an orthorhombic crystal containing a single RANKL trimer in the ASU. Collectively, these diffraction-quality crystals provide 8 independent views of the RANKL monomer. The structure of RANKL was phased by molecular replacement and refined to a resolution of 2.6 Å. With the exception of some mobile loop regions, the entire RANKL ectodomain, minus residues 158-161 of the amino terminus, is well ordered in the final crystal structure (crystallographic R factor 23.5% and R_{free} 28.0%, FIG. 1). The RANKL trimer

The core β-strands of RANKL adopts a fold that is characteristic of the TNF family cytokines. Each RANKL monomer consists of a -sandwich, composed of two flat antiparallel β-pleated sheets. The first sheet is formed by β-strands A, H, C, and F, while the second sheet is formed by β-strands B, G, D, and E (FIGS. 2A-2D, FIG. 3). The inner AHCF β-sheet is involved in intersubunit association, whereas the BGDE β-sheet contributes largely to the outer surface. RANKL monomers self-associate along a three-fold rotational axis of symmetry. The trimeric complex is best described as a truncated pyramid, with the membrane-proximal base wider than the distal apex. The RANKL trimer is 55 Å high, with approximate diameters of 55 Å at the base and 35 Å at the apex. The homotrimer is assembled such that one edge of the β-sandwich in each RANKL monomer, strands E and F, packs against the inner hydrophobic face of the AHCF β-sheet of the neighboring monomer (FIGS. 2A, 2C). As such, the trimeric interface of the RANKL homotrimer consists of a platform of edge-to-face interactions. Viewed from the side, each monomer adopts a slight left-handed twist around the rotational axis (FIGS. 2A, 2B). While the amino acid composition of each RANKL monomer is identical, the trimer is not topologically equivalent by crystallographic symmetry.

RANKL monomers bury approximately 6099.5 Å² of solvent accessible surface upon trimerization (2042.1 Å² for monomer X, 2043.2 Å² for Y, and 2014.2 Å² for Z), as compared to other TNF family cytokines—6591.3 Å² for TRAIL, 5670.6 Å² for CD40L, 5600.8 Å² for TNFα, 5826.9 Å² for TNFβ, and 5130.0 Å² for ACRP30. The trimeric interface constitutes approximately 26% of the solvent accessible surface of each monomer. RANKL does not form heterotrimers with other TNF family members. The selectivity of this interaction is dictated by sequence-specific intersubunit hydrogen bonding. Each RANKL molecule contains 43 such buried polar interactions, while the typical TNF family cytokine possesses an average of 26 intersubunit hydrogen bonds per molecule. Although hydrophobic interactions do not engender specificity, they constitute a major driving force for self-association. Of the residues involved in RANKL monomer trimerization, 40% are hydrophobic (FIG. 3). Almost all of the amino acid residues that participate in intersubunit interactions reside within the 10 highly conserved -strands, leaving structurally divergent surface loops accessible for receptor-ligand interactions (FIG. 3).

The solvent-accessible surface loops of RANKL are unique within the TNF family, displaying markedly divergent lengths and conformations: the AA″ loop (residues 170-193) bridges β-strands A and A″, the CD loop (residues 224-233), the DE loop (residues 245-251), and the EF loop (residues 261-269). In particular, RANKL possesses a longer AA″ loop and a shorter EF loop than the typical TNF family member. This divergence is best appreciated by superimposing the structure of RANKL over those of TNF and TRAIL (FIG. 2E). The internal -strands that constitute the TNF core fold exhibit nearly identical topology, while the loops that interconnect these strands fail to superimpose. Representative electron density of the -strand core of RANKL is presented in FIG. 2F.

Thus, applicants have elucidated the mechanism of binding between RANKL and RANK, thereby identifying an essential part of a defined binding site responsible for the binding between these molecules which play key roles in RANK-mediated signaling. Furthermore, applicants have utilized the RANKL crystal structure, to provide further insights into the processes of RANKL-RANK interaction. Applicants have thereby provided the essential data by which compounds, compositions and methods for the prevention and treatment of osteoporosis and other conditions.

Thus, applicants' method utilizes the discovery of this molecular mechanism of RANKL-RANK protein binding to identify an essential part of a defined binding site responsible for RANK biology.

### Peptide Compounds

Thus, the present invention is directed to compounds which mimic the capability of RANKL to bind RANK molecules, thereby activating this receptor. Specifically, applicants have shown the unique structure of RANKL which permits the binding RANKL and RANK

In a preferred embodiment of the invention, the mimic compounds are peptides or peptide analogs that are capable of binding RANK. As will be apparent from the structural information provided herein by applicants, the core sequence of such peptides and peptide analogs may be derived from the unique RANKL structural motifs. The core sequence may correspond identically to the sequence of a RANKL binding motif, or it may include one or more substitutions, preferably conservative substitutions, and/or insertions and/or deletions which do not negatively the binding of such peptide to RANK or its activation thereby.

Moreover, the core sequence may be flanked at either of both of its N- and/or C-termini by residues of random sequence (i.e., sequences that do not necessarily correspond to other portions of the RANKL ectodomain from which the core sequence is derived). When included, such flanking residues should not significantly alter the ability of the core sequence to bind and activate RANK. Thus, typically the compounds of the invention will include fewer than 5 flanking residues at each terminus, preferably fewer than 3 flanking residues, and most preferably no flanking residues.

Such molecules may contain a number of modifications known to those skilled in the art. For instance, substituted amide linkages may generally include, but are not limited to, groups of the formula —C(O)N(R)—, where R is (C₁-C₆) alkyl, substituted (C₁-C₆) alkyl, (C₂-C₆) alkenyl, substituted (C₂-C₆) alkenyl, (C₂-C₆) alkynyl, substituted (C₂-C₆) alkynyl, (C₅-C₂₀) aryl, substituted (C₅-C₂₀) aryl, (C₆-C₂₆) arylalkyl, substituted (C₆-C₂₆) arylalkyl, 5-20 membered heteroaryl, substituted 5-20 membered heteroaryl, 6-26 membered heteroarylalkyl and substituted 6-26 membered heteroarylalkyl.

Isosteres of amide linkages may generally include, but are not limited to, —CH₂NH—, —CH₂S—, —CH₂CH₂—, —CH═CH— (cis and trans), —C(O)CH₂—, —CH(OH)CH₂— and —CH₂SO—. Compounds having such non-amide linkages and methods for preparing such compounds are well-known in the art (see, e.g., Spatola, Vega Data. 1:3(1983); Spatola, “Peptide Backbone Modifications” In: Chemistry and Biochemistry of Amino Acids Peptides and Proteins, Weinstein, ed., Marcel Dekker, New York, p. 267 (general review) (1983)); Morley, Trends Pharm. Sci. 1:463-468(1980); Hudson, et al., Int. J. Prot. Res. 14:177-185(1979) (—CH₂NH—, —CH₂CH₂—); Spatola, et al., Life Sci. 38:1243-1249(1986) (—CH₂—S); Hann, J. Chem. Soc. Perkin Trans. I. 1:307-314(1982) (—CH═CH—, cis and trans); Almquist, et al., J. Med. Chem. 23:1392-1398(1980) (—0COCH₂—); Jennings-White, et al., Tetrahedron. Lett. 23:2533 (—COCH₂—); European Patent Application EP 45665 (1982) CA 97:39405 (—CH(OH)CH₂—); Holladay, et al., Tetrahedron Lett. 24:4401-4404(1983) (—C(OH)CH₂—); and Hruby, Life Sci. 31:189-199(1982) (—CH₂—S—).

Additionally, one or more amide linkages can be replaced with peptidomimetic or amide mimetic moieties which do not significantly interfere with the structure or activity of the peptides. Suitable amide mimetic moieties are described, for example, in Olson, et al., J. Med. Chem. 36:3039-3049(1993).

Compounds comprising RANKL mimics that are peptide analogs may provide significant therapeutic advantages, as their non-peptide interlinkages may confer the compound with enhanced stability towards proteases and/or peptidases, thereby conferring the compounds with increases in vivo stability compared to a corresponding peptide.

Additionally, the various residues may be selected from amongst the genetically encoded amino acids, as well as from genetically non-encoded amino acids. Moreover, the residues may be in either the D- or L-configuration, as long as the compound retains activity. Compounds including D-amino acids may have enhanced in vivo stability.

The peptides and peptide analogs may optionally include, in addition to the RANKL mimic sequence, a 1 to 5 residue peptide or peptide analog at either or both termini. Peptide analogs typically contain at least one modified interlinkage, such as a substituted amide or an isostere of an amide, as described above. Such additional peptides or peptide analogs may have an amino acid sequence derived from another portion of the RANKL amino acid sequence or, alternatively, their sequences may be completely random. Compounds including such random sequences may be tested for biological activity in the various assays and methods described in a later section.

The residues which comprise such additional peptides or peptide analogs may be genetically encoded or non-encoded, and may be in either the D- or L-configuration. In one embodiment, when the sequence defined by formula (I) is a peptide, one or both termini are “capped” with 1 to 5 residue peptides composed wholly of D-amino acids that serve to protect the core sequence from degradation in vivo by proteases and/or peptidases.

Also included within the scope of the present invention are “blocked” forms of the peptides and peptide analogs in which the N- and/or C-terminus is blocked with a moiety capable of reacting with the N-terminal —NH₂ or C-terminal —C(O)OH. Such blocked compounds are typically N-terminal acylated and/or C-terminal amidated or esterified. Typical N-terminal blocking groups include R₁C(O)—, where R₁ is hydrogen, (C₁-C₆) alkyl, (C₂-C₆) alkenyl, (C₂-C₆) alkynyl, (C₅-C₂₀) aryl, (C₆-C₂₆) arylalkyl, 5-20 membered heteroaryl or 6-26 membered heteroarylalkyl. Preferred N-terminal blocking groups include acetyl, formyl and dansyl. Typical C-terminal blocking groups include —C(O)NR₁R₁ and —C(O)OR₁, where each R1 is independently as defined above. Preferred C-terminal blocking groups include those in which each R₁ is independently (C₁-C₆) alkyl, preferably methyl, ethyl, propyl or isopropyl.

It will be appreciated that by virtue of the present invention, the above-described polypeptides can be synthesized using conventional synthesis procedures commonly used by one skilled in the art. For example, the polypeptides can be chemically synthesized using an automated peptide synthesizer (such as one manufactured by Pharmacia LKB Biotechnology Co., LKB Biolynk 4170 or Milligen, Model 9050 (Milligen, Millford, Mass.)) following the method of Sheppard, et al., Journal of Chemical Society Perkin I, p. 538 (1981). In this procedure, N,N′-dicyclohexylcarbodiimide is added to amino acids whose amine functional groups are protected by 9-flourenylmethoxycarbonyl (Fmoc) groups and anhydrides of the desired amino acids are produced. These Fmoc-amino acid anhydrides can then be used for peptide synthesis. A Fmoc-amino acid anhydride corresponding to the C-terminal amino acid residue is fixed to Ultrosyn A resin through the carboxyl group using dimethylaminopyridine as a catalyst. Next, the resin is washed with dimethylformamide containing piperidine, and the protecting group of the amino functional group of the C-terminal acid is removed. The next amino acid corresponding to the desired peptide is coupled to the C-terminal amino acid. The deprotecting process is then repeated. Successive desired amino acids are fixed in the same manner until the peptide chain of the desired sequence is formed. The protective groups other than the acetoamidomethyl are then removed and the peptide is released with solvent.

Alternatively, the polypeptides can be synthesized by using nucleic acid molecules which encode the peptides of this invention in an appropriate expression vector which include the encoding nucleotide sequences. Such DNA molecules may be prepared using an automated DNA sequencer and the well-known codon-amino acid relationship of the genetic code. Such a DNA molecule also may be obtained as genomic DNA or as cDNA using oligonucleotide probes and conventional hybridization methodologies. Such DNA molecules may be incorporated into expression vectors, including plasmids, which are adapted for the expression of the DNA and production of the polypeptide in a suitable host such as bacterium, e.g., Escherichia coli, yeast cell, mammalian cell, or insect cell.

It is known that certain modifications can be made without completely abolishing the polypeptide's activity. Modifications include the removal and addition of amino acids. Polypeptides containing other modifications can be synthesized by one skilled in the art and compounds comprising such polypeptides may be tested for biological activity in the various assays and methods described in a later section. Thus, the effectiveness of the polypeptides can be modulated through various changes in the amino acid sequence or structure.

Further, it should be understood that the mimic may be modified using methods known in the art to improve binding, specificity, solubility, safety, or efficacy. A necessary characteristic of these preferred compounds is the capability to interact with RANK during its activation. The compound can be any compound, preferably a peptide, which has the ability to mimic RANKL's binding to, and activation of, RANK.

### Crystalline RANKL Complexes

The present invention provides high-resolution three-dimensional structure and atomic structure coordinates of crystalline complexes of the ectodomain of RANKL as determined by X-ray crystallography. The atomic structure coordinates of crystalline RANKL complexes, obtained to 2.6 Å resolution, are listed in FIG. 1.

Additional RANK-binding compounds can be modeled and synthesized utilizing the atomic coordinates obtained from the resolution of the crystal structure of RANKL, particularly when combined with applicants' discoveries using RANKL mutants as to unique surface loops and binding clefts formed by the RANKL homotrimers. For example, as discussed herein, applicants utilized the crystal structure and mutant data to identify the three spatially distinct, but equivalent receptor-binding regions (see, e.g., FIG. 2C) which are essential components required for interaction with RANK.

Thus, the invention encompasses crystals of the RANKL ectodomain. In a preferred embodiment, the crystalline form of the polypeptides corresponding to the RANKL ectodomain complexes effectively diffracts X-rays for the determination of the atomic coordinates of the complex to a resolution of from about 4 Å to about 2.6 Å or greater.

Preferably, crystals of the invention comprise crystallized polypeptides corresponding to complexes of the wild-type RANKL ectodomain. The crystals of the invention include native crystals in which the crystallized complex is substantially pure as well as heavy-atom atom derivative crystals in which the crystallized complex is in association with one or more heavy-metal atoms.

It is to be understood that the crystalline complex from which the atomic structure coordinates of the invention can be obtained is not limited to the wild-type RANKL. Indeed, the crystals may comprise mutants of wild-type RANKL. Mutants of wild-type RANKL are obtained by replacing at least one amino acid residue in the sequence of the RANKL ectodomain a different amino acid residue, or by adding or deleting one or more amino acid residues within the wild-type sequence and/or at its N- and/or C-terminus.

The types of mutants contemplated by this invention include conservative mutants, non-conservative mutants, deletion mutants, truncated mutants, extended mutants, methionine mutants, selenomethionine mutants, cysteine mutants and selenocysteine mutants. A mutant may have, but need not have, RANK binding activity. Preferably, a mutant displays biological activity that is substantially similar to that of the wild-type RANKL polypeptide. Methionine, selenomethione, cysteine, and selenocysteine mutants are particularly useful for producing heavy-atom derivative crystals, as described in detail, below.

It will be recognized by one of skill in the art that the types of mutants contemplated herein are not mutually exclusive; that is, for example, a polypeptide having a conservative mutation in one amino acid may in addition have a truncation of residues at the N-terminus, and several Leu or lie→Met mutations.

Structure based sequence alignments of polypeptides in a protein family or of homologous polypeptide domains can be used to identify potential amino acid residues in the polypeptide sequence that are candidates for mutation. Identifying mutations that do not significantly interfere with the three-dimensional structure of RANKL, particularly its specificity to bind RANK and/or that do not deleteriously affect, and that may even enhance, the activity of the peptide will depend, in part, on the region where the mutation occurs.

Conservative amino acid substitutions are well known in the art, and include substitutions made on the basis of a similarity in polarity, charge, solubility, hydrophobicity and/or the hydrophilicity of the amino acid residues involved. Typical conservative substitutions are those in which the amino acid is substituted with a different amino acid that is a member of the same class or category, as those classes are defined herein. Thus, typical conservative substitutions include aromatic to aromatic, apolar to apolar, aliphatic to aliphatic, acidic to acidic, basic to basic, polar to polar, etc. Other conservative amino acid substitutions are well known in the art. It will be recognized by those of skill in the art that the amino acids in the wild-type polypeptide sequence can be conservatively substituted with other amino acids without deleteriously affecting the biological activity and/or three-dimensional structure of the molecule, provided that such substitutions do not involve residues that are critical for activity, as discussed above.

The heavy-atom derivative crystals from which the atomic structure coordinates of the invention alternatively may be obtained generally comprise a crystalline complex in association with one or more heavy metal atoms. The polypeptides may correspond to a wild-type or a mutant RANKL complex, which may optionally be further associated with one or more molecules. There are two types of heavy-atom derivatives of polypeptides: heavy-atom derivatives resulting from exposure of the proteins to a heavy metal in solution, wherein crystals are grown in medium comprising the heavy metal, or in crystalline form, wherein the heavy metal diffuses into the crystal, and heavy-atom derivatives wherein at least one of the polypeptides in the complex comprises heavy-atom containing amino acids, e.g., selenomethionine and/or selenocysteine mutants.

In practice, heavy-atom derivatives of the first type can be formed by soaking a native crystal in a solution comprising heavy metal atom salts, or organometallic compounds, e.g., lead chloride, gold thiomalate, thimerosal, uranyl acetate, platinum tetrachloride, osmium tetraoxide, zinc sulfate, and cobalt hexamine, which can diffuse through the crystal and bind to the crystalline polypeptides.

Heavy-atom derivatives of this type can also be formed by adding to a crystallization solution comprising the polypeptides to be crystallized an amount of a heavy metal atom salt, which may associate with and be incorporated into the crystal. The location(s) of the bound heavy metal atom(s) can be determined by X-ray diffraction analysis of the crystal. This information, in turn, may be used to generate the phase information needed to construct the three-dimensional structure of the proteins in the complex.

The native and/or heavy-atom derivative crystals from which the atomic structure coordinates of the invention are obtained can be obtained by conventional means as are well-known in the art of protein crystallography, including batch, liquid bridge, dialysis, and vapor diffusion methods (see, e.g., McPherson, Preparation and Analysis of Protein Crystals, John Wiley, New York(1982); McPherson, Eur. J. Biochem. 189:1-23.(1990); Weber, Adv. Protein Chem. 41:1-36.(1991)). Generally, native crystals are grown by dissolving substantially pure polypeptide encoding for the RANKL ectodomain complex in an aqueous buffer containing a precipitant at a concentration just below that necessary to precipitate the protein. Examples of precipitants include, but are not limited to, polyethylene glycol, ammonium sulfate, 2-methyl-2,4-pentanediol, sodium citrate, sodium chloride, glycerol, isopropanol, lithium sulfate, sodium acetate, sodium formate, potassium sodium tartrate, ethanol, hexanediol, ethylene glycol, dioxane, t-butanol and combinations thereof. Water is removed by controlled evaporation to produce precipitating conditions, which are maintained until crystal growth ceases.

In a preferred embodiment, native crystals are grown by vapor diffusion in hanging drops (McPherson, Preparation and Analysis of Protein Crystals, John Wiley, New York(1982); McPherson, Eur. J. Biochem. 189:1-23.(1990)). In this method, the polypeptide/precipitant solution is allowed to equilibrate in a closed container with a larger aqueous reservoir having a precipitant concentration optimal for producing crystals. Generally, less than about 25 μl of substantially pure RANKL polypeptide solution is mixed with an equal volume of reservoir solution, giving a precipitant concentration about half that required for crystallization. This solution is suspended as a droplet underneath a coverslip, which is sealed onto the top of the reservoir. The sealed container is allowed to stand, usually for about 2-6 weeks, until crystals grow.

Heavy-atom derivative crystals can be obtained by soaking native crystals in mother liquor containing salts of heavy metal atoms. Further, heavy-atom derivative crystals can also be obtained from SeMet and/or SeCys mutants, as described above for native crystals.

Mutant proteins may crystallize under slightly different crystallization conditions than wild-type protein, or under very different crystallization conditions, depending on the nature of the mutation, and its location in the protein. For example, a non-conservative mutation may result in alteration of the hydrophilicity of the mutant, which may in turn make the mutant protein either more soluble or less soluble than the wild-type protein. Typically, if a protein becomes more hydrophilic as a result of a mutation, it will be more soluble than the wild-type protein in an aqueous solution and a higher precipitant concentration will be needed to cause it to crystallize. Conversely, if a protein becomes less hydrophilic as a result of a mutation, it will be less soluble in an aqueous solution and a lower precipitant concentration will be needed to cause it to crystallize. If the mutation happens to be in a region of the protein involved in crystal lattice contacts, crystallization conditions may be affected in more unpredictable ways.

The dimensions of a unit cell of a crystal are defined by six numbers, the lengths of three unique edges, a, b, and c, and three unique angles α, β, and γ. The type of unit cell that comprises a crystal is dependent on the values of these variables. In one embodiment, the crystal of the RANKL ectodomain complex has the space group of P2₁2₁2₁ with unit cell dimensions of a=65.3 Å±0.2 Å, b=82.0 Å±0.2 Å and c=99.5 Å±0.2 Å such that the three dimensional structure of the crystallized complex can be determined to a resolution of from about 3 Å to about 2.6 Å or greater. In another embodiment, the crystals of the RANKL ectodomain complex has the space group R3 with unit cell dimensions of a=b=150.6 Å±0.2 Å and c=139.5 Å±0.2 Å such that the three-dimensional structure of the complex can be determined to a resolution of from about 4.0 Å to about 3.0 Å or greater.

When a crystal is placed in an X-ray beam, the incident X-rays interact with the electron cloud of the molecules that make up the crystal, resulting in X-ray scatter. The combination of X-ray scatter with the lattice of the crystal gives rise to nonuniformity of the scatter; areas of high intensity are called diffracted X-rays. The angle at which diffracted beams emerge from the crystal can be computed by treating diffraction as if it were reflection from sets of equivalent, parallel planes of atoms in a crystal (Bragg's Law). The most obvious sets of planes in a crystal lattice are those that are parallel to the faces of the unit cell. These and other sets of planes can be drawn through the lattice points. Each set of planes is identified by three indices, hkl. The h index gives the number of parts into which the a edge of the unit cell is cut, the k index gives the number of parts into which the b edge of the unit cell is cut, and the I index gives the number of parts into which the c edge of the unit cell is cut by the set of hkl planes. Thus, for example, the 235 planes cut the a edge of each unit cell into halves, the b edge of each unit cell into thirds, and the c edge of each unit cell into fifths. Planes that are parallel to the bc face of the unit cell are the 100 planes; planes that are parallel to the ac face of the unit cell are the 010 planes; and planes that are parallel to the ab face of the unit cell are the 001 planes.

When a detector is placed in the path of the diffracted X-rays, in effect cutting into the sphere of diffraction, a series of spots, or reflections, are recorded to produce a “still” diffraction pattern. Each reflection is the result of X-rays reflecting off one set of parallel planes, and is characterized by an intensity, which is related to the distribution of molecules in the unit cell, and hkl indices, which correspond to the parallel planes from which the beam producing that spot was reflected. If the crystal is rotated about an axis perpendicular to the X-ray beam, a large number of reflections is recorded on the detector, resulting in a diffraction pattern.

The unit cell dimensions and space group of a crystal can be determined from its diffraction pattern. First, the spacing of reflections is inversely proportional to the lengths of the edges of the unit cell. Therefore, if a diffraction pattern is recorded when the X-ray beam is perpendicular to a face of the unit cell, two of the unit cell dimensions may be deduced from the spacing of the reflections in the x and y directions of the detector, the crystal-to-detector distance, and the wavelength of the X-rays. Those of skill in the art will appreciate that, in order to obtain all three unit cell dimensions, the crystal must be rotated such that the X-ray beam is perpendicular to another face of the unit cell. Second, the angles of a unit cell can be determined by the angles between lines of spots on the diffraction pattern. Third, the absence of certain reflections and the repetitive nature of the diffraction pattern, which may be evident by visual inspection, indicate the internal symmetry, or space group, of the crystal. Therefore, a crystal may be characterized by its unit cell and space group, as well as by its diffraction pattern.

Once the dimensions of the unit cell are determined, the likely number of polypeptides in the asymmetric unit can be deduced from the size of the polypeptide, the density of the average protein, and the typical solvent content of a protein crystal, which is usually in the range of 30-70% of the unit cell volume.

The diffraction pattern is related to the three-dimensional shape of the molecule by a Fourier transform. The process of determining the solution is in essence a re-focusing of the diffracted X-rays to produce a three-dimensional image of the molecule in the crystal. Since re-focusing of X-rays cannot be done with a lens at this time, it is done via mathematical operations.

The sphere of diffraction has symmetry that depends on the internal symmetry of the crystal, which means that certain orientations of the crystal will produce the same set of reflections. Thus, a crystal with high symmetry has a more repetitive diffraction pattern, and there are fewer unique reflections that need to be recorded in order to have a complete representation of the diffraction. The goal of data collection, a dataset, is a set of consistently measured, indexed intensities for as many reflections as possible. A complete dataset is collected. if at least 80%, preferably at least 90%, most preferably at least 95% of unique reflections are recorded. In one embodiment, a complete dataset is collected using one crystal. In another embodiment, a complete dataset is collected using more than one crystal of the same type.

Sources of X-rays include, but are not limited to, a rotating anode X-ray generator such as a Rigaku RU-200 or a beamline at a synchrotron light source, such as the Advanced Photon Source at Argonne National Laboratory. Suitable detectors for recording diffraction patterns include, but are not limited to, X-ray sensitive film, multiwire area detectors, image plates coated with phosphorus, and CCD cameras. Typically, the detector and the X-ray beam remain stationary, so that, in order to record diffraction from different parts of the crystal's sphere of diffraction, the crystal itself is moved via an automated system of moveable circles called a goniostat.

One of the biggest problems in data collection, particularly from macromolecular crystals having a high solvent content, is the rapid degradation of the crystal in the X-ray beam. In order to slow the degradation, data is often collected from a crystal at liquid nitrogen temperatures. In order for a crystal to survive the initial exposure to liquid nitrogen, the formation of ice within the crystal must be prevented by the use of a cryoprotectant. Suitable cryoprotectants include, but are not limited to, low molecular weight polyethylene glycols, ethylene glycol, sucrose, glycerol, xylitol, and combinations thereof. Crystals may be soaked in a solution comprising the one or more cryoprotectants prior to exposure to liquid nitrogen, or the one or more cryoprotectants may be added to the crystallization solution. Data collection at liquid nitrogen temperatures may allow the collection of an entire dataset from one crystal. Once a dataset is collected, the information is used to determine the three-dimensional structure of the molecule in the crystal. However, this cannot be done from a single measurement of reflection intensities because certain information, known as phase information, is lost between the three-dimensional shape of the molecule and its Fourier transform, the diffraction pattern. This phase information must be acquired by methods described below in order to perform a Fourier transform on the diffraction pattern to obtain the three-dimensional structure of the molecule in the crystal. It is the determination of phase information that in effect refocuses X-rays to produce the image of the molecule.

One method of obtaining phase information is by isomorphous replacement, in which heavy-atom derivative crystals are used. In this method, the positions of heavy atoms bound to the molecules in the heavy-atom derivative crystal are determined, and this information is then used to obtain the phase information necessary to elucidate the three-dimensional structure of a native crystal (Blundel, et al., Protein Crystallography, Academic Press (1976)).

Another method of obtaining phase information is by molecular replacement, which is a method of calculating initial phases for a new crystal of a polypeptide or polypeptide complex whose structure coordinates are unknown by orienting and positioning a polypeptide whose structure coordinates are known within the unit cell of the new crystal so as to best account for the observed diffraction pattern of the new crystal. Phases are then calculated from the oriented and positioned polypeptide and combined with observed amplitudes to provide an approximate Fourier synthesis of the structure of the molecules comprising the new crystal. (Lattman, Methods in Enzymology. 115:55-77(1985); Rossmann, “The Molecular Replacement Method,” Int. Sci. Rev. Ser. No. 13, Gordon & Breach, New York(1 972).

A third method of phase determination is multi-wavelength anomalous dispersion (MAD). In this method, X-ray diffraction data are collected at several different wavelengths from a single crystal containing at least one heavy atom with absorption edges near the energy of incoming X-ray radiation. The resonance between X-rays and electron orbitals leads to differences in X-ray scattering that permits the locations of the heavy atoms to be identified, which in turn provides phase information for a crystal of a polypeptide. A detailed discussion of MAD analysis can be found in Hendrickson, Trans. Am. Crystallogr. Assoc., 21:11(1985); Hendrickson, et al., EMBO J. 9:1665(1990); and Hendrickson, Science 4:91(1991).

A fourth method of determining phase information is single wavelength anomalous dispersion or SAD. In this technique, X-ray diffraction data are collected at a single wavelength from a single native or heavy-atom derivative crystal, and phase information is extracted using anomalous scattering information from atoms such as sulfur or chlorine in the native crystal or from the heavy atoms in the heavy-atom derivative crystal. The wavelength of X-rays used to collect data for this phasing technique need not be close to the absorption edge of the anomalous scatterer. A detailed discussion of SAD analysis can be found in Brodersen, et al., Acta Cryst. D56:431441(2000).

A fifth method of determining phase information is single isomorphous replacement with anomalous scattering or SIRAS. This technique combines isomorphous replacement and anomalous scattering techniques to provide phase information for a crystal of a polypeptide. X-ray diffraction data are collected at a single wavelength, usually from a single heavy-atom derivative crystal. Phase information obtained only from the location of the heavy atoms in a single heavy-atom derivative crystal leads to an ambiguity in the phase angle, which is resolved using anomalous scattering from the heavy atoms. Phase information is therefore extracted from both the location of the heavy atoms and from anomalous scattering of the heavy atoms. A detailed discussion of SIRAS analysis can be found in North, Acta Cryst. 18:212-216(1965); Matthews, Acta Cryst. 20:82-86(1966).

Once phase information is obtained, it is combined with the diffraction data to produce an electron density map, an image of the electron clouds that surround the molecules in the unit cell. The higher the resolution of the data, the more distinguishable are the features of the electron density map, e.g., amino acid side chains and the positions of carbonyl oxygen atoms in the peptide backbones, because atoms that are closer together are resolvable. A model of the macromolecule is then built into the electron density map with the aid of a computer, using as a guide all available information, such as the polypeptide sequence and the established rules of molecular structure and stereochemistry. Interpreting the electron density map is a process of finding the chemically realistic conformation that fits the map precisely.

After a model is generated, a structure is refined. Refinement is the process of minimizing the function φ, which is the difference between observed and calculated intensity values (measured by an R-factor), and which is a function of the position, temperature factor, and occupancy of each non-hydrogen atom in the model. This usually involves alternate cycles of real space refinement, i.e., calculation of electron density maps and model building, and reciprocal space refinement, i.e., computational attempts to improve the agreement between the original intensity data and intensity data generated from each successive model. Refinement ends when the function φ converges on a minimum wherein the model fits the electron density map and is stereochemically and conformationally reasonable. During refinement, ordered solvent molecules are added to the structure.

The atomic structure coordinates and machine-readable media of the invention have a variety of uses. The present invention encompasses the structure coordinates and other information, e.g., amino acid sequence, connectivity tables, vector-based representations, temperature factors, etc., used to generate the three-dimensional structures of the polypeptides for use in the software programs described below and other software programs. For example, the coordinates are useful for solving the three-dimensional X-ray diffraction and/or solution structures of other proteins, including mutant RANKL ectodomain complexes, complexes that are further associated with other molecules, and unrelated proteins, to high resolution. Structural information may also be used in a variety of molecular modeling and computer-based screening applications to, for example, intelligently design mutants of the crystallized RANKL ectodomain complex that have altered biological activity and to computationally design and identify compounds that bind RANK. Such compounds may be used as lead compounds in pharmaceutical efforts to identify compounds that modulate RANK signaling as a therapeutic approach toward the treatment of several types of diseases mediated or associated with loss of bone mass or bone defects.

In a further aspect of the invention, such potential compounds are evaluated for their capacity to bind RANK and enhance bone formation. These methods comprise designing and synthesizing candidate compounds using the atomic coordinates of the three dimensional structure of such crystals and associated structural data and screening them for the ability to bind RANK and/or to enhance RANK modulation. The RANK modulating activity of the compound is determined by assaying the compounds for their binding affinity for RANK or for increased presence of markers of bone formation activity. Such compounds which are able to bind RANK and enhance bone formation can be used in pharmaceutical compositions directed toward therapeutic treatments related to prevention or prophylaxis of bone loss.

Additionally, the invention encompasses machine-readable media embedded with the three-dimensional structures of the models described herein, or with portions thereof. As used herein, “machine readable medium” refers to any medium that can be read and accessed directly by a computer or scanner. Such media include, but are not limited to: magnetic storage media, such as floppy discs, hard disc storage medium and magnetic tape; optical storage media such as optical discs or CD-ROM; electrical storage media such as RAM or ROM; and hybrids of these categories such as magnetic/optical storage media. Such media further include paper on which is recorded a representation of the atomic structure coordinates, e.g., Cartesian coordinates, that can be read by a scanning device and converted into a three-dimensional structure with an Optical Character Recognition (OCR).

A variety of data storage structures are available to a skilled artisan for creating a computer readable medium having recorded thereon the atomic structure coordinates of the invention or portions thereof and/or X-ray diffraction data. The choice of the data storage structure will generally be based on the means chosen to access the stored information. In addition, a variety of data processor programs and formats can be used to store the sequence and X-ray data information on a computer readable medium. Such formats include, but are not limited to, Protein Data Bank (“PDB”) format (Research Collaboratory for Structural Bioinformatics;

http://www.rcsb.org/pdb/docs/format/pdbguide2.2/guide2.2_frame.html); Cambridge Crystallographic Data Centre format (http://www.ccdc.cam.ac.uk/support/csd_doc/volume3/z323.html); Structure-data (“SD”) file format (MDL Information Systems, Inc.; Dalby, et al., J. Chem. Inf. Comp. Sci. 32:244-255(1992)), and line-notation, e.g., as used in SMILES (Weininger, J. Chem. Inf. Comp. Sci. 28:31-36(1988)). Methods of converting between various formats read by different computer software will be readily apparent to those of skill in the art, e.g., BABEL (v. 1.06, Walters & Stahl, ©1992,1993, 1994; hftp://www.brunel.ac.uk/departments/chem/babel.htm.) All format representations of the polypeptide coordinates described herein, or portions thereof, are contemplated by the present invention. By providing computer readable medium having stored thereon the atomic coordinates of the invention, one of skill in the art can routinely access the atomic coordinates of the invention, or portions thereof, and related information for use in modeling and design programs, described in detail below.

While Cartesian coordinates are important and convenient representations of the three-dimensional structure of a polypeptide, those of skill in the art will readily recognize that other representations of the structure are also useful. Therefore, the three-dimensional structure of a polypeptide, as discussed herein, includes not only the Cartesian coordinate representation, but also all alternative representations of the three-dimensional distribution of atoms. For example, atomic coordinates may be represented as a Z-matrix, wherein a first atom of the protein is chosen, a second atom is placed at a defined distance from the first atom, a third atom is placed at a defined distance from the second atom so that it makes a defined angle with the first atom. Each subsequent atom is placed at a defined distance from a previously placed atom with a specified angle with respect to the third atom, and at a specified torsion angle with respect to a fourth atom. Atomic coordinates may also be represented as a Patterson function, wherein all interatomic vectors are drawn and are then placed with their tails at the origin. This representation is particularly useful for locating heavy atoms in a unit cell. In addition, atomic coordinates may be represented as a series of vectors having magnitude and direction and drawn from a chosen origin to each atom in the polypeptide structure. Furthermore, the positions of atoms in a three-dimensional structure may be represented as fractions of the unit cell (fractional coordinates), or in spherical polar coordinates.

Additional information, such as thermal parameters, which measure the motion of each atom in the structure, chain identifiers, which identify the particular chain of a multi-chain protein or protein complex in which an atom is located, and connectivity information, which indicates to which atoms a particular atom is bonded, is also useful for representing a three-dimensional molecular structure.

### Uses of the Atomic Structure Coordinates

Structure information, typically in the form of the atomic structure coordinates, can be used in a variety of computational or computer-based methods to, for example, design, screen for and/or identify compounds that bind the crystallized polypeptide or a portion or fragment thereof, or to intelligently design mutants that have altered biological properties.

In one embodiment, the crystals and structure coordinates obtained therefrom are useful for identifying and/or designing compounds that bind RANK as an approach towards developing new therapeutic agents. For example, a high resolution X-ray structure will often show the locations of ordered solvent molecules around the protein, and in particular at or near putative binding sites on the protein. This information can then be used to design molecules that bind these sites, the compounds synthesized and tested for binding in biological assays (Travis, Science. 262:1374(1993)).

In yet another embodiment, the structures can be used to computationally screen small molecule data bases for chemical entities or compounds that can bind in whole, or in part, to RANK or RANKL. In this screening, the quality of fit of such entities or compounds to the binding site may be judged either by shape complementarity or by estimated interaction energy (Meng, et al., J. Comp. Chem. 13:505-524(1992)).

The design of compounds that bind to RANK or RANKL according to this invention generally involves consideration of two factors. First, the compound must be capable of physically and structurally associating with RANK or RANKL. This association can be covalent or non-covalent. For example, covalent interactions may be important for designing suicide or irreversible inhibitors of a protein. Non-covalent molecular interactions important in the association of RANKL with RANK include hydrogen bonding, ionic interactions and van der Waals and hydrophobic interactions. Second, the compound must be able to assume a conformation that allows it to associate with RANK or RANKL. Although certain portions of the compound will not directly participate in this association with the protein, those portions may still influence the overall conformation of the molecule. This, in turn, may have a significant impact on potency. Such conformational requirements include the overall three-dimensional structure and orientation of the chemical group or compound in relation to all or a portion of the binding site, or the spacing between functional groups of a compound comprising several chemical groups that directly interact with the protein.

The potential inhibitory or binding effect of a chemical compound on RANK or RANKL may be analyzed prior to its actual synthesis and testing by the use of computer modeling techniques. If the theoretical structure of the given compound suggests insufficient interaction and association between it and the protein, synthesis and testing of the compound is unnecessary. However, if computer modeling indicates a strong interaction, the molecule may then be synthesized and tested for its ability to bind to the protein and inhibit its activity. In this manner, synthesis of ineffective compounds may be avoided.

A RANKL mimic may be computationally evaluated and designed by means of a series of steps in which chemical groups or fragments are screened and selected for their ability to associate with the individual binding pockets or interface surfaces of each of the proteins. One skilled in the art may use one of several methods to screen chemical groups or fragments for their ability to associate with RANK. This process may begin by visual inspection of, for example, the protein/protein interfaces or the binding site on the computer screen based on the crystal complex coordinates. Selected fragments or chemical groups may then be positioned in a variety of orientations, or docked, at an individual surface of RANKL that participates in a protein/protein interface or in the binding pocket. Docking may be accomplished using software such as QUANTA and SYBYL, followed by energy minimization and molecular dynamics with standard molecular mechanics forcefields, such as CHARMM and AMBER.

Specialized computer programs may also assist in the process of selecting fragments or chemical groups. These include:

1. GRID (Goodford, J. Med. Chem. 28:849-857(1985)). GRID is available from Oxford University, Oxford, UK;

2. MCSS (Miranker & Karplus, Proteins: Structure, Function and Genetics. 11:29-34(1991)). MCSS is available from Molecular Simulations, Burlington, Mass.;

3. AUTODOCK (Goodsell & Olsen, Proteins: Structure, Function, and Genetics 8:195-202(1990)). AUTODOCK is available from Scripps Research Institute, La Jolla, Calif.; and

4. DOCK (Kuntz, et al., J. Mol. Biol. 161:269-288(1982)). DOCK is available from University of California, San Francisco, Calif.

Once suitable chemical groups or fragments have been selected, they can be assembled into a single compound or inhibitor. Assembly may proceed by visual inspection of the relationship of the fragments to each other in the three-dimensional image displayed on a computer screen in relation to the structure coordinates of. This would be followed by manual model building using software such as QUANTA or SYBYL.

Useful programs to aid one of skill in the art in connecting the individual chemical groups or fragments include:

1. CAVEAT (Bartlett, et al., ‘CAVEAT: A Program to Facilitate the Structure-Derived Design of Biologically Active Molecules’(1989). In Molecular Recognition in Chemical and Biological Problems', Special Pub., Royal Chem. Soc. 78:182-196). CAVEAT is available from the University of California, Berkeley, Calif.;

2. 3D Database systems such as MACCS-3D (MDL Information Systems, San Leandro, Calif.). This area is reviewed in Martin, J. Med. Chem. 35:2145-2154(1992)); and

3. HOOK (available from Molecular Simulations, Burlington, Mass.).

Instead of proceeding to build a RANKL mimic, in a step-wise fashion one fragment or chemical group at a time, as described above, such compounds may be designed as a whole or ‘de novo’ using either an empty binding site or the surface of a protein that participates in protein/protein interactions or optionally including some portion(s) of a known activator(s). These methods include:

1. LUDI (Bohm, J. Comp. Aid. Molec. Design 6:61-78(1992)). LUDI is available from Molecular Simulations, Inc., San Diego, Calif.;

2. LEGEND (Nishibata & Itai, Tetrahedron 47:8985(1991)). LEGEND is available from Molecular Simulations, Burlington, Mass.; and

3. LeapFrog (available from Tripos, Inc., St. Louis, Mo.).

Other molecular modeling techniques may also be employed in accordance with this invention. See, e.g., Cohen, et al., J. Med. Chem. 33:883-894(1990). See also, Navia & Murcko, Current Opinions in Structural Biology 2:202-210(1992).

Once a compound has been designed or selected by the above methods, the efficiency with which that compound may bind to RANK or RANKL may be tested and optimized by computational evaluation. For example, a compound that has been designed or selected to function as a RANKL mimic must also preferably occupy a volume not overlapping the volume occupied by the RANKL -binding site residues when RANK is bound. An effective mimic must preferably demonstrate a relatively small difference in energy between its bound and free states (i.e., it must have a small deformation energy of binding). Thus, the most efficient inhibitors should preferably be designed with a deformation energy of binding of not greater than about 10 kcal/mol, preferably, not greater than 7 kcal/mol. Inhibitors may interact with the protein in more than one conformation that is similar in overall binding energy. In those cases, the deformation energy of binding is taken to be the difference between the energy of the free compound and the average energy of the conformations observed when the inhibitor binds to the protein.

A compound selected or designed for binding to RANK may be further computationally optimized so that in its bound state it would preferably lack repulsive electrostatic interaction with the target protein. Such non-complementary electrostatic interactions include repulsive charge-charge, dipole-dipole and charge-dipole interactions. Specifically, the sum of all electrostatic interactions between the inhibitor and the protein when the mimic is bound to it preferably make a neutral or favorable contribution to the enthalpy of binding.

Specific computer software is available in the art to evaluate compound deformation energy and electrostatic interaction. Examples of programs designed for such uses include: Gaussian 92, revision C (Frisch, Gaussian, Inc., Pittsburgh, Pa. ©1992); AMBER, version 4.0 (Kollman, University of California at San Francisco, ©1994); QUANTA/CHARMM (Molecular Simulations, Inc., Burlington, Mass., ©1994); and Insight II/Discover (Biosym Technologies Inc., San Diego, Calif., ©1994). These programs may be implemented, for instance, using a computer workstation, as are well-known in the art. Other hardware systems and software packages will be known to those skilled in the art.

Once a RANKL mimic compound has been optimally selected or designed, as described above, substitutions may then be made in some of its atoms or chemical groups in order to improve or modify its binding properties. Generally, initial substitutions are conservative, i.e., the replacement group will have approximately the same size, shape, hydrophobicity and charge as the original group. One of skill in the art will understand that substitutions known in the art to alter conformation should be avoided. Such altered chemical compounds may then be analyzed for efficiency of binding to RANK by the same computer methods described in detail above.

Because such complexes may crystallize in more than one crystal form, the structure coordinates of RANKL complexes, or of portions thereof, are particularly useful to solve the structure of those other crystal forms. They may also be used to solve the structure of mutants, RANKL further complexed to another molecule, or of the crystalline form of any other protein or protein complex with significant amino acid sequence homology to any functional domain of RANKL. Similarly, the structure coordinates, are particularly useful to solve the structure of other co-crystal forms of RANKL and its receptors. They may also be used to solve the structure of mutants, of a RANKL-RANK co-complex, or of the crystalline form of any other protein or protein co-complex with significant amino acid sequence homology to any functional domain of RANKL.

One method that may be employed for this purpose is molecular replacement. In this method, the unknown crystal structure, whether it is another crystal form of a RANKL complex, a mutant, a RANKL-RANK or RANKL-OPG complex or co-complex that is further complexed to another molecule, or the crystal of some other protein or protein co-complex with significant amino acid sequence homology to any functional domain of one of the proteins in the co-complex crystal, may be determined using phase information from the RANKL complex structure coordinates. This method will provide an accurate three-dimensional structure for the unknown protein or protein co-complex in the new crystal more quickly and efficiently than attempting to determine such information ab initio.

If an unknown crystal form has the same space group as and similar cell dimensions to the known co-complex crystal form, then the phases derived from the known crystal form can be directly applied to the unknown crystal form, and in turn, an electron density map for the unknown crystal form can be calculated. Difference electron density maps can then be used to examine the differences between the unknown crystal form and the known crystal form. A difference electron density map is a subtraction of one electron density map, e.g., that derived from the known crystal form, from another electron density map, e.g., that derived from the unknown crystal form. Therefore, all similar features of the two electron density maps are eliminated in the subtraction and only the differences between the two structures remain. Similarly, if amino acid side chains have different conformations in the two crystal forms, then those differences will be highlighted by peaks (positive electron density) and valleys (negative electron density) in the difference electron density map, making the differences between the two crystal forms easy to detect. However, if the space groups and/or cell dimensions of the two crystal forms are different, then this approach will not work and molecular replacement must be used in order to derive phases for the unknown crystal form.

All of the complexes referred to above may be studied using well-known X-ray diffraction techniques and may be refined versus 1.5 Å or higher to 3 Å resolution X-ray date to an R value of about 0.20 or less using computer software, such as X-PLOR (Yale University, ©1992, distributed by Molecular Simulations, Inc.). See, e.g., Blundel, et al., Protein Crystallography, Academic Press(1976); Methods in Enzymology, vol. 114 & 115, Wyckoff, et al., eds., Academic Press, 1985. This information may thus be used to optimize known classes and more importantly, to design and synthesize novel classes of RANKL mimics

Subsets of the atomic structure coordinates can be used in any of the above methods. Particularly useful subsets of the coordinates include, but are not limited to, coordinates of residues lining an active site, coordinates of residues that participate in important protein-protein contacts at an interface, and Cα coordinates. For example, the coordinates of one domain of a protein that contains the active site may be used to design mimics that bind to that site, even though the protein is fully described by a larger set of atomic coordinates. Therefore, as described in detail for the specific embodiments, below, a set of atomic coordinates that define the entire polypeptide chain, although useful for many applications, do not necessarily need to be used for the methods described herein.

### Uses of Subsets of Atomic Coordinates in Specific Embodiments

The structure coordinates of the present invention, and subsets thereof, are useful for designing or screening for compounds that mimic RANKL. The high resolution X-ray structures of the RANKL complexes of the present invention show details of its unique interfacial residues, particularly when combined with the functional information derived from applicants' structure-based mutagenesis analysis. This information can be used to design and/or screen for compounds that bind to the sites of RANK interaction.

In one embodiment, the RANKL ectodomain coordinates, or the subset of such coordinates that are the residues in the groove regions of the RANKL homotrimers, where the RANK interacts with RANK are useful for designing and/or screening for compounds that bind in the groove. A subset of structure coordinates of RANKL useful in this embodiment of the invention include those as set forth in FIG. 3.

The following examples illustrate the invention, but are not to be taken as limiting the various aspects of the invention so illustrated.

### Protein Expression and Purification

The biologically-active extracellular domain of RANKL was expressed in Escherichia coli as a GST-fusion protein. Briefly, a cDNA encoding the core ectodomain of murine RANKL residues 158-316 was cloned into pGEX-6P-I (Amersham Pharmacia Biotech, Piscataway, N.J.) downstream of glutathione S-transferase (GST) using the SalI and NotI restriction endonucleases. Following IPTG (isopropylthio-β-D-galactoside)-mediated induction of protein expression in protease-deficient BL21 (DE3) E. coli, cells were triturated into a nondenaturing lysis buffer comprising 150 mM NaCl, 20 mM Tris-HCl pH 8.0, and 1 mM EDTA. Lysates were incubated with glutathione sepharose (Amersham Pharmacia Biotech) for affinity purification of the fusion protein, followed by excessive washing with buffer comprising 150 mM NaCl and 20 mM Tris-HCl pH 8.0. The GST-fusion protein was released from the affinity matrix by competitive elution with glutathione. The isolated protein was then subjected to anion exchange, followed by dialysis against 150 mM NaCl, 20 mM Tris-HCl, pH 7.2. Separation of RANKL from its GST fusion partner was accomplished by proteolytic cleavage with the type 14 human rhinovirus 3C protease (Amersham Pharmacia Biotech), performed for 4 hours at 4° C. in 50 mM Tris-HCl, pH 7.0, 150 mM NaCl, 10 mM EDTA, and 1 mM DTT. Uncleaved fusion protein, as well as the GST-tagged protease, was removed by passage over a glutathione affinity matrix. The final product was further purified by size exclusion chromatography on Superdex200 (Amersham Pharmacia Biotech), in which it migrated with a molecular weight of 55 kD, consistent with the size of a homotrimer. Identity was continued by mass spectrometry, with a mass of 19,034 Da conforming to predicted values ±1 Da. Purified RANKL was concentrated to 20 mg/ml in 20 mM NaCl, 20 mM Tris-HCl, pH 8.0.

### Crystallization and Data Collection

Crystals of RANKL were grown by vapor diffusion in hanging drops at 20° C. Diffraction quality crystals were obtained with a precipitant comprising 80 mM HEPES pH 7.5,16 mM CaCl₂, and 11-14% PEG 4000. Two crystal forms predominated: primitive rhombohedral crystals (space group R3) growing to a typical size 0.4 mm×0.4 mm×0.2 mm, with unit cell dimensions a=b=150.6 Å×c=139.5 Å, containing 7 RANKL monomers in the asymmetric unit (ASU); and primitive orthorhombic crystals (space group P2₁2₁2₁) growing to a typical size of 0.3 mm×0.3 mm×0.4 mm, with unit cell dimensions a=65.3 Å×b=82.0 Å× and c=99.5 Å, containing 3 RANKL monomers in the ASU. Crystals were cryoprotected in 20% glycerol and flash cooled in liquid N₂. Complete data to 3.0 Å resolution was collected for the rhombohedral crystal and 2.6 Å for the orthorhombic crystal by the oscillation method (1.5°) using a Rigaku rotating anode generator with CuKα radiation, equipped with Yale mirrors and an R-Axis IV detector. Data sets were processed with DENZO and SCALEPACK.

### Structure Determination and Refinement

The RANKL structure was determined initially by molecular replacement using models constructed from TNFβ (PDB code 1TNR) and TRAIL (PDB code 1D4V) coordinates. These structures were combined into a chimeric search model by deletion of divergent regions and incorporation of RANKL sequence using the Insight II program module Homology (Molecular Simulations Inc., San Diego, Calif.). Translation function searches using AMORE (Navaza, J., et al., Methods Enzymol. 276:581-591(1997)) yielded 7 RANKL monomers in the ASU of the rhombohedral crystal, arranged as two trimers with a seventh monomer situated on the crystallographic three-fold axis of symmetry. Rigid body refinement of the chimeric model against the data set gave an R factor of 46% for all data between 20-4 Å resolution. The initial RANKL model was built into seven-fold averaged electron density maps calculated at 3 Å resolution. Model building into composite 2FoFc omit maps were carried out using the program O (Jones, T. A., et al., Acta Crystallogr. A. 47:110-119(1991)). Iterative rounds of model building and refinement using both strict and restrained NCS operators were carried out using CNS (33). Phases for orthorhombic RANKL were obtained by molecular replacement with the rhombohedral coordinates. Translation functions were run in all possible primitive orthorhombic enantiomorphs, with the highest signal unambiguously in space group P2₁2₁2₁. Rigid body refinement of the three monomers situated in the ASU yielded an R factor of 37% for 20-4 Å resolution data. Model building was initiated on maps calculated using noncrystallographic three-fold averaging, and atomic refinement was carried out using restrained NCS operators, excluding structurally diverse loop regions of each monomer.

### Structural Analysis

Atomic accessible area was calculated with NACCESS v2.1.1 (Hubbard, S. J. & Thornton, J. M., University College, London.). Briefly, a 1.4 Å radius probe was rolled over the van der Waal's surface of RANKL and other TNF family cytokine structures for calculation of solvent accessible surface areas. HBPLUS v3.0 (McDonald, I. K., et al., J. Mol. Biol. 238:777-793(1994)) was used to assess neighbor interactions and the geometry of hydrogen bonds.

### Mutagenesis

Site-directed mutagenesis of murine RANKL was performed by polymerase chain reaction. Mutagenized RANKL constructs were expressed in E. coli as described above, with confirmation of identity by nucleic acid sequencing and mass spectrometry. Homotrimerization, as indicated by migration on size exclusion chromatography, was taken as evidence for proper protein folding and assembly. Lack of endotoxin contamination was confirmed by limulus amoebocyte lysate (LAL) assay (BioWhittaker, Walkersville, Md.).

### Cell Culture

Pure populations of murine osteoclast precursors were isolated from bone marrow described in Lam, J., et al., J. Clin. Invest. 106:1481-1488(2000). Cells were cultured in α-MEM containing 10% fetal bovine serum, with addition of 10 ng/ml macrophage colony stimulating factor (M-CSF) and various concentrations of RANKL to induce osteoclast differentiation. Cells were incubated at 37° C. in a humidified atmosphere containing 6% CO₂. and supplemented with fresh media and cytokines daily. Typical mature osteoclast formation was observed between culture days 5 and 9.

### Cytochemistry

Osteoclast formation was identified histochemically by the method of Burstone (Burstone, M. S., J. Natl. Cancer Inst. 20:601-606(1958)). Briefly, cellular acid phosphatase activity was employed to cleave the chromogenic substrate naphthol AS-BI phosphate in the presence of 0.05 M sodium tartrate. Quantitation of tartrate-resistant acid phosphatase (TRAP) activity was accomplished by addition of a colorimetric substrate, 5.5 mM p-nitrophenyl phosphate, in the presence of 10 mM sodium tartrate at pH 4.5. The reaction product was quantified by spectroscopic measurement of optical absorbance at 405 nm.

### Structure Determination

The biologically active ectodomain of murine RANKL (residues 158-316, QPFA . . . QDID), expressed recombinantly in E. coli crystallizes in two forms: a rhombohedral crystal containing seven monomers in the ASU, arranged as two trimers with a seventh monomer situated on the crystallographic three-fold axis of symmetry; and an orthorhombic crystal containing a single RANKL trimer in the ASU. Collectively, these diffraction-quality crystals provide 8 independent views of the RANKL monomer. The structure of RANKL was phased by molecular replacement and refined to a resolution of 2.6 Å. With the exception of some mobile loop regions, the entire RANKL ectodomain, minus residues 158-161 of the amino terminus, is well-ordered in the final crystal structure (crystallographic R factor 23.5% and R_{free} 28.0%, FIG. 1).

### The RANKL Trimer

The core β-strands of RANKL adopt a fold that is characteristic of the TNF family cytokines. Each RANKL monomer consists of a β-sandwich, composed of two flat antiparallel β-pleated sheets. The first sheet is formed by β-strands A, H, C, and F, while the second sheet is formed by β-strands B, G, D, and E (FIGS. 2A-2D, FIG. 3). The inner AHCF β-sheet is involved in intersubunit association, whereas the BGDE β-sheet contributes largely to the outer surface. RANKL monomers self-associate along a three-fold rotational axis of symmetry. The trimeric complex is best described as a truncated pyramid, with the membrane-proximal base wider than the distal apex. The RANKL trimer is 55 Å high, with approximate diameters of 55 Å at the base and 35 Å at the apex. The homotrimer is assembled such that one edge of the β-sandwich in each RANKL monomer, strands E and F, packs against the inner hydrophobic face of the AHCF β-sheet of the neighboring monomer (FIGS. 2A, 2C). As such, the trimeric interface of the RANKL homotrimer consists of a platform of edge-to-face interactions. Viewed from the side, each monomer adopts a slight left-handed twist around the rotational axis (FIGS. 2A, 2B). While the amino acid composition of each RANKL monomer is identical, the trimer is not topologically equivalent by crystallographic symmetry.

RANKL monomers bury approximately 6099.5 Å² of solvent accessible surface upon trimerization (2042.1 Å² for monomer X, 2043.2 Å² for Y, and 2014.2 Å² for Z), as compared to other TNF family cytokines—6591.3 Å² for TRAIL, 5670.6 Å² for CD40L, 5600.8 Å² for TNFα, 5826.9 Å² for TNFβ, and 5130.0 Å² for ACRP30. The trimeric interface constitutes approximately 26% of the solvent accessible surface of each monomer. RANKL does not form heterotrimers with other TNF family members. The selectivity of this interaction is dictated by sequence-specific intersubunit hydrogen bonding. Each RANKL molecule contains 43 such buried polar interactions, while the typical TNF family cytokine possesses an average of 26 intersubunit hydrogen bonds per molecule. Although hydrophobic interactions do not engender specificity, they constitute a major driving force for self-association. Of the residues involved in RANKL monomer trimerization, 40% are hydrophobic (FIG. 3). Almost all of the amino acid residues that participate in intersubunit interactions reside within the 10 highly conserved β-strands, leaving structurally divergent surface loops accessible for receptor-ligand interactions (FIG. 3).

The solvent-accessible surface loops of RANKL are unique within the TNF family, displaying markedly divergent lengths and conformations: the AA″ loop (residues 170-193) bridges β-strands A and A″, the CD loop (residues 224-233), the DE loop (residues 245-251), and the EF loop (residues 261-269). In particular, RANKL possesses a longer AA″ loop and a shorter EF loop than the typical TNF family member. This divergence is best appreciated by superimposing the structure of RANKL over those of TNF and TRAIL (FIG. 2E). The internal β-strands that constitute the TNF core fold exhibit nearly identical topology, while the loops that interconnect these strands fail to superimpose. Representative electron density of the β-strand core of RANKL is presented in FIG. 2F.

### Receptor Binding Sites

The two receptors for RANKL, namely RANK and OPG, belong to the TNFR family. Based on the two known crystal structures for TNF family receptor-ligand complexes, it is accepted that TNF family cytokines bind one elongated receptor molecule along each of the three clefts formed by neighboring monomers of the homotrimer. These intersubunit grooves can be appreciated looking downwards along the three-fold axis of rotational symmetry (FIGS. 2C, 2D). From this perspective, it is apparent that the cleft is formed by two adjacent monomers of the RANKL homotrimer, thus ligand homotrimerization is requisite for receptor binding. In this manner, the RANKL trimer exhibits three spatially distinct, but equivalent, receptor-binding regions. Importantly, portions of the structurally unique AA″, CD, DE, and EF loops of RANKL line the sides of the receptor binding cleft (FIG. 2C). Many naturally-occurring, as well as synthesized TNF family cytokine variants exhibit altered affinities in receptor binding that result from mutations in these loops (FIG. 3), which have been demonstrated to directly contact receptor molecules in the crystal structures of the TRAIL-DR5 complex (Hymowitz, S. G., et al., 5. Mol. Cell. 4:563-571(1999), Mongkolsapaya, J., et al., Nat Struct Biol. 6:1048-1053(1999), Cha, S. S., et al., J. Biol. Chem. 275:31171-31177(2000)) and the TNFβ-TNFR complex (Banner, D. W., et al., Cell. 73:431-445(1993)).

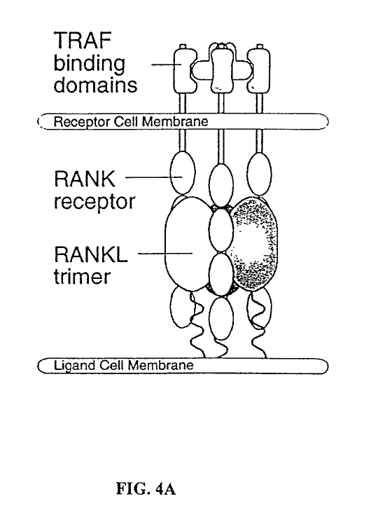

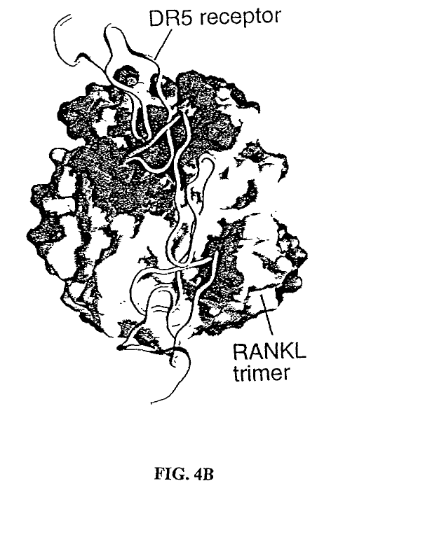

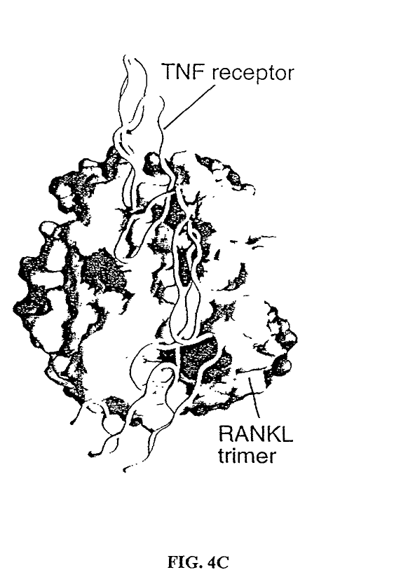

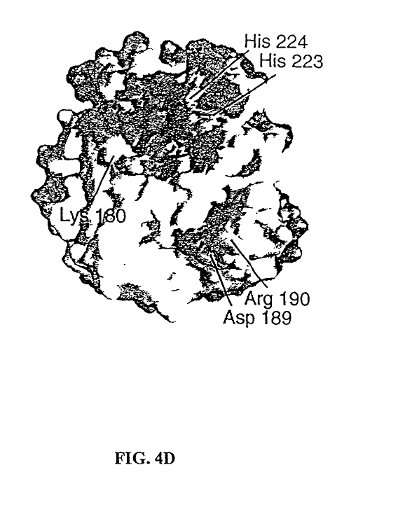

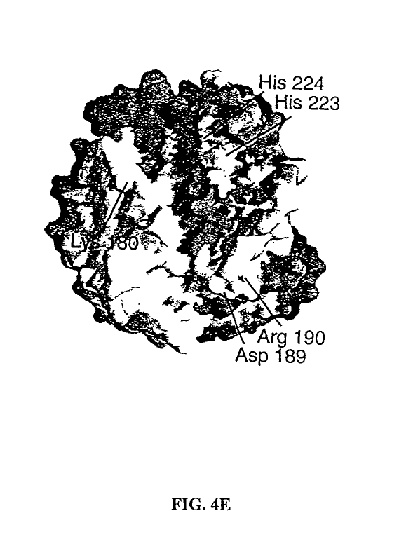

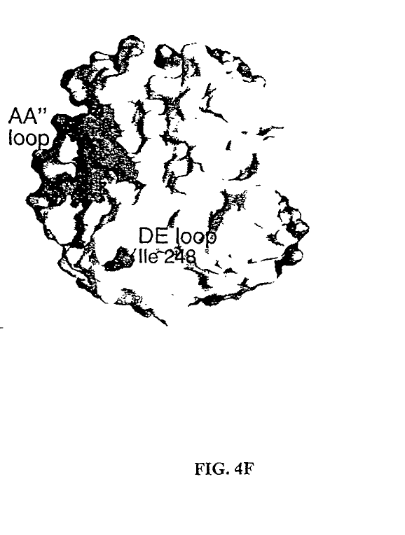

Two crystal structures of TNF family cytokines in complex with their receptors have been reported, TRAIL-DR5 and TNFβ TNFR. These structures provide a framework for modeling the receptor-ligand interactions of other TNF family members. Although DR5 and TNFR share only 16 of 130 non-cysteine extracellular residues, their ligand binding domains occupy nearly equivalent positions in their respective complexes. Superposition of the Cα trace of the DR5 ligand-binding domain onto that of TNFR results in a root mean squared deviation (rmsd) of only 0.7 Å. Thus, superposition of RANKL in place of the ligands in these complexes should allow a reasonable prediction of interfacial residues on RANKL. Such analysis was used to identify residues of RANKL that are positioned within 4 Å of DR5 or TNFR (FIG. 3 and FIGS. 4A-4C). Due to the remarkable structural congruence in the ligand-binding domains of these receptors, it is a reasonable inference that these residues may similarly serve as interfacial residues for RANK and OPG. When both the DR5 and TNFR contacts are mapped onto the surface of RANKL, it is apparent that prospective contact sites for both receptors (FIG. 4D) encompass structurally unique elements of RANKL. Furthermore, when sequence conservation is also mapped onto the surface of RANKL, it can be appreciated that portions of the receptor contact patches employed by both receptors (yellow shading in FIG. 4D) are not conserved across the family (white shading in FIG. 4E). This observation fits well with the highly specific recognition known to occur among TNF family cytokines and their receptors.

### Structure-based Mutagenesis

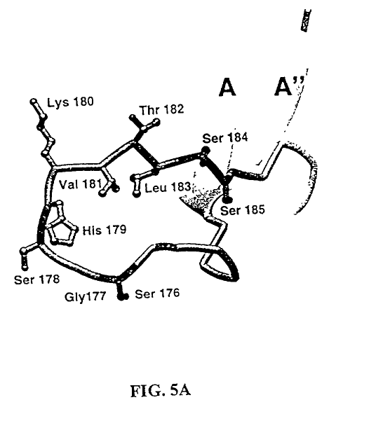

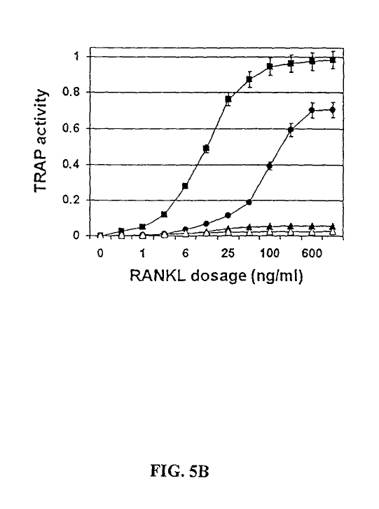

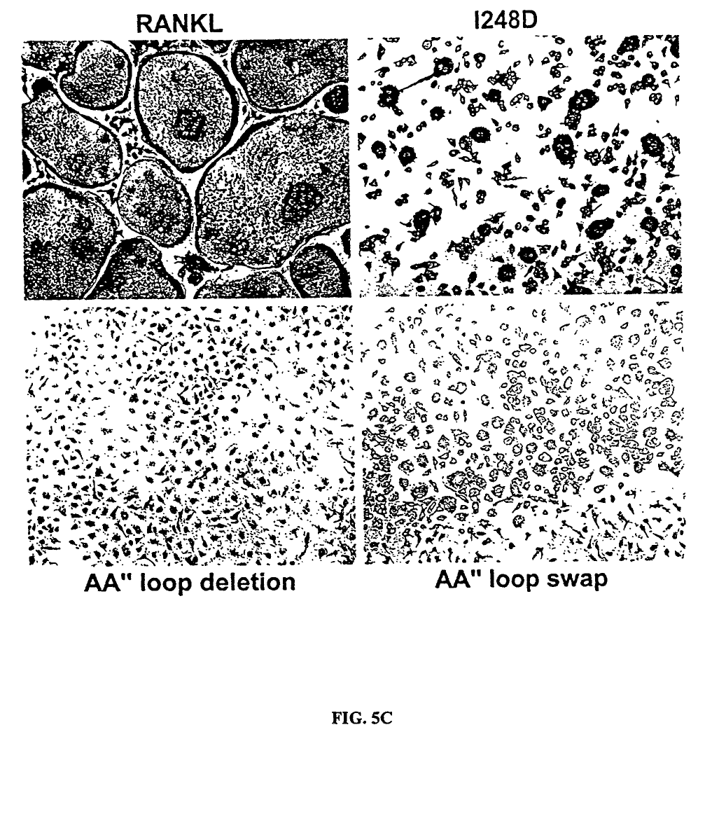

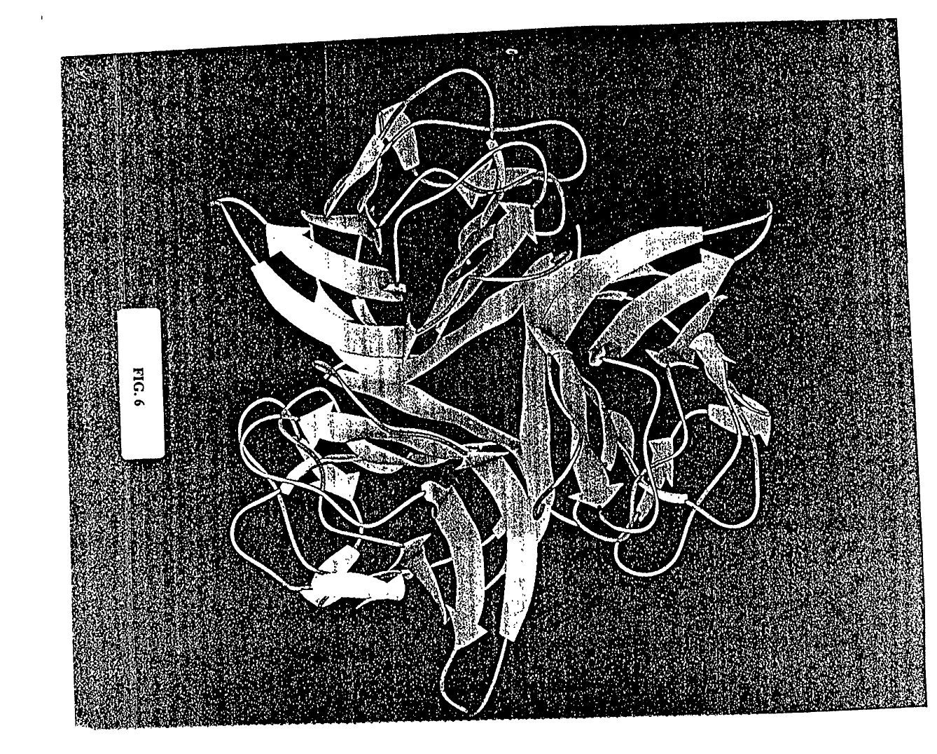

Comparison with known TNF-TNFR structures suggests that RANKL may bind its receptors through its unique solvent accessible loops. We tested the biological relevance of these predictions by performing structure-based mutagenesis experiments. Three mutations were generated that impact the biological function of RANKL: a single amino acid substitution in the DE loop, replacing Ile₂₄₈ with Asp (1₂₄₈D); a deletion of the AA″ loop, from Gly₁₇₇ to Leu₁₈₃ (AA″ loop deletion); and a replacement of the AA″ loop of RANKL (177-183) with that of TNF (AA″ loop swap) (FIG. 5A). RANKL, bearing AA″ loop deletion and loop swap mutants, fails to induce osteoclast precursors to differentiate in vitro, whereas the I₂₄₈D substitution mutant demonstrates an 8-fold decrease in potency relative to native RANKL (FIG. 5B). Furthermore, the osteoclasts induced by I₂₄₈D RANKL exhibit dysfunctional morphology, namely mononuclearity and small size relative to multinucleated, well-spread osteoclasts typically induced by native RANKL (FIG. 5C). As such, the AA″ loop deletion and AA″ loop swap mutants of RANKL fail to induce cellular differentiation via RANK. In contrast, construct I₂₄₈D represents a novel form of RANKL that activates RANK, but induces comparable levels of cellular differentiation only when administered at 8 times the concentration of native RANKL.

Other features, objects and advantages of the present invention will be apparent to those skilled in the art. The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the present invention.

What is claimed is:

1. A composition comprising a protein complex in crystalline form, wherein said complex comprises an amino acid sequence of a RANKL ectodomain.

2. The composition of claim 1, wherein said crystal is of diffraction quality.

3. The composition of claim 1, wherein said crystal is a native crystal.

4. The composition of claim 1, wherein said crystal is a heavy-atom derivative crystal.

5. The composition of claim 1, wherein said amino acid sequence comprises an extracellular domain of RANKL.

6. The composition of claim 5, wherein said crystal is of diffraction quality.

7. The composition of claim 5, wherein said crystal is a native crystal.

8. The composition of claim 5, wherein said crystal is a heavy-atom derivative crystal.

9. The composition of claim 1, wherein said composition comprises a primitive rhombohedral crystal comprising seven RANKL monomers in an asymmetric unit.

10. The composition of claim 9, wherein said crystal is of diffraction quality.

11. The composition of claim 9, wherein said crystal is a native crystal.

12. The composition of claim 9, wherein said crystal is a heavy-atom derivative crystal.

13. The composition of claim 9, wherein the crystal has a space group of R3 with unit cell dimensions of a=b=150.6 ∈±0.2 ∈ and c=139.5 ∈±0.2 ∈.

14. The composition of claim 13, wherein said crystal is of diffraction quality.

15. The composition of claim 13, wherein said crystal is a native crystal.

16. The composition of claim 13, wherein said crystal is a heavy-atom derivative crystal.

17. The composition of claim 1, wherein said composition comprises a primitive orthorhombic crystal comprising three RANK monomers in an asymmetric unit.

18. The composition of claim 17, wherein the crystal has a space group of P2₁2₁2₁ with unit cell dimensions of a=65.3 ∈±0.2 ∈, b=82.0 ∈±0.2 ∈ and c=99.5 ∈±0.2 Å.

19. The composition of claim 17, wherein said crystal is of diffraction quality.

20. The composition of claim 17, wherein said crystal is a native crystal.

21. The composition of claim 17, wherein said crystal is a heavy-atom derivative crystal.

22. The composition of claim 1, wherein the protein is a RANKL mutant.

23. The crystal of claim 22, wherein the mutant is a selenomethionine or selenocysteine mutant.

24. The crystal of claim 22, wherein the mutant is a conservative mutant.

25. The crystal of claim 22, wherein the mutant is a truncated or extended mutant.

26. The composition of claim 1, wherein said crystal is produced by a method comprising the steps of:
(a) mixing a volume of a solution comprising the RANKL ectodomain with a volume of a reservoir solution comprising a precipitant; and
(b) incubating the mixture obtained in step (a) over the reservoir solution in a closed container, under conditions suitable for crystallization until the crystal forms.

27. A method of crystallizing a RANKL protein complex, said method comprising:
(a) mixing a volume of a solution comprising the RANKL protein complex with a volume of a reservoir solution comprising a precipitant; and
(b) incubating the mixture obtained in step (a) over the reservoir solution in a closed container, under conditions suitable for crystallization until the crystal forms.

28. A method of identifying a compound with RANK modulating activity, comprising the step of using a three-dimensional structural representation of a RANKL ectodomain crystal complex, or a fragment thereof comprising a binding cleft, to computationally screen a candidate compound for an ability to bind a RANK binding site.

29. The method of claim 28, further comprising the steps of:
(a) synthesizing the candidate compound; and
(b) screening the candidate compound for osteogenetic activity.

30. The method of claim 27, wherein the three-dimensional structural information comprises the atomic structure coordinates of a RANKL ectodomain.

31. The method of claim 30, wherein the three-dimensional structural information further comprises the atomic structure coordinates of residues comprising a receptor binding cleft of RANKL.

32. The method of claim 31, wherein the atomic structure coordinates of residues comprising the receptor binding cleft of RANKL are obtained from the atomic structure coordinates of a RANKL ectodomain complex.

33. A method of identifying a RANK or OPG modulating compound comprising the step of using a three-dimensional structural representation of a RANKL ectodomain complex, or a fragment thereof comprising a receptor binding cleft of RANKL, to computationally design a synthesizable candidate compound that binds RANK OPG or RANKL.

34. The method of claim 33, wherein the computational design comprises the steps of:
(a) identifying chemical entities or fragments capable of mimicking the receptor binding cleft of RANKL; and
(b) assembling the chemical entities or fragments into a single molecule to provide the structure of the candidate compound.

35. The method of claim 34, further including the steps of:
(a) synthesizing the candidate compound; and
(b) screening the candidate compound for modulating RANK activity.

36. The method of claim 35, wherein the structural information comprises the atomic structure coordinates of a RANKL ectodomain.

37. The method of claim 36, wherein the structural information further comprises atomic structure coordinates of residues comprising a receptor binding cleft of RANKL.

38. A machine-readable medium embedded with information that corresponds to a three-dimensional structural representation of a crystalline RANKL ectodomain complex or a fragment or portion thereof.

39. The machine-readable medium of claim 38, in which the information comprises atomic structure coordinates, or a subset thereof.

40. The machine-readable medium of claim 38, wherein the RANKL ectodomain complex comprises a rhombohedral crystal comprising seven monomers in an asymmetric unit.

41. The machine-readable medium of claim 40, in which the information comprises atomic structure coordinates, or a subset thereof.

42. The machine-readable medium of claim 38, wherein at least one monomer of the complex is a mutant.

43. The machine-readable medium of claim 42, in which the information comprises atomic structure coordinates, or a subset thereof.

44. The machine-readable medium of claim 38, wherein the RANKL ectodomain complex comprises an orthorhombic crystal comprising three RANKL monomers in the asymmetric unit.

45. The machine-readable medium of claim 44, wherein at least one monomer of the complex is a mutant.

46. The machine-readable medium of claim 45, in which the information comprises atomic structure coordinates, or a subset thereof.

47. The machine-readable medium of claim 44, in which the information comprises atomic structure coordinates, or a subset thereof.

48. A compound comprising a non-natural mutation in the DE loop of RANKL.

49. The mutant of claim 48, wherein the mutation is in residue 248.

50. The mutant of claim 49, comprising an I₂₄₈D substitution.

51. A compound comprising a non-natural mutation in the AA″ loop of RANKL.

52. The mutant of claim 51, wherein the AA″ loop mutation comprises a deletion of the AA″ loop from Gly₁₇₇ to Leu₁₈₃.

53. The mutant of claim 51, wherein the AA″ loop mutation comprises a substitution of RANKL residues 177-183 with that of TNF.
